(19) United States
(12) Patent Application Publication
(10) Pub. No.: US 2016/0221932 A1
(43) Pub. Date: Aug. 4, 2016

(54) Methods and Uses of Melatonin Ligands

(71) Applicants: Sergio DOMINGUEZ LOPEZ, San Antonio, TX (US); Gabriella GOBBI, Montreal (CA)

(72) Inventors: Sergio Dominguez Lopez, San Antonio, TX (US); Gabriella Gobbi, Montréal (CA)

(21) Appl. No.: 14/912,143

(22) PCT Filed: Aug. 15, 2014
(86) PCT No.: PCT/CA2014/000624

§ 371 Date: Feb. 15, 2016

Related U.S. Application Data
(60) Provisional application No. 61/866,193, filed on Aug. 15, 2013.

(51) Int. Cl. C07C 233/36

(52) U.S. Cl. CPC C07C 233/36

(57) ABSTRACT

The disclosure provides methods and uses for alleviating and/or treating pain including pain disorders using melatonin ligands of Formula I: or pharmaceutically acceptable salts thereof wherein: n is 1 or 2; m is 0, 1 or 2; p is 0, 1, 2, 3, 4, 5, 6, 7 or 8; v is 2 or 3; A is aryl or heteroaryl; Z is 0, S or NR₈; Y is chosen from hydrogen, aryl, heteroaryl, C₁-C₆ alkyl, C₃-C₆ cycloalkyl, and R is chosen from hydrogen, hydroxyl, —OCF₃, CF₃, C₁-C₈ alkyl, C₁-C₈ alkyloxy, C₁-C₈ alkylthio, halogen and —Z—(CH₂ₚ-A; R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl; R₂ is chosen from hydrogen, C₁-C₄ alkyl, C₁-C₄ alkyloxy, OCF₃, CF₃, hydroxyl, and halogen; R₃ is chosen from hydrogen, C₁-C₄ alkyl, C₁-C₄ alkyloxy, OCF₃, CF₃, hydroxyl and halogen; R and R₃ may be connected together to form an -0-(CH₂)ᵥ bridge representing with the carbon atoms to which they are attached a 5- or 6-membered heterocyclic ring system; R₄ is chosen from hydrogen, C₁-C₄ alkyl, C₁-C₄ alkyloxy, OCF₃, CF₃, hydroxyl, and halogen; R₆ is chosen from hydrogen and C₁-C₄ alkyl; R₇ is chosen from hydrogen, C₁-C₄ alkyl, C₁-C₄ alkyloxy, OCF₃, CF₃, hydroxyl and halogen; and R₈ is chosen from hydrogen and C₁-C₄ alkyl.

### FIELD

The present disclosure relates to methods and uses of melatonin ligands and more particularly to melatonin ligands having analgesic, antinociceptive, antiallodynic, anesthetic and antihyperalgesic properties.

### BACKGROUND

An important challenge in medical care is the treatment of pain, in particular, in those patients suffering from injury and post operatory acute pain or patients suffering chronic pain associated with a disease such as cancer, or neuropathic pain originated by a neuronal lesion (Campbell and Meyer, 2006). Acute pain is a predictable response to an insult associated with surgery, trauma, or acute illness. It usually decreases over a period of few minutes, hours, days or weeks. Chronic pain is variously defined as pain lasting more than a month. Chronic pain is a major health problem that afflicts a significant number of patients, resulting in personal suffering, reduced productivity and substantial health care costs. The neuronal nature of pain signaling (or nociception) makes its treatment difficult; potent drugs are necessary to affect or block pain neuronal pathways and they usually affect other physiological functions (Stone and Molliver, 2009).

The neurohormone melatonin (N-acetyl-5-methoxytryptamine, MLT) is secreted by the pineal gland during the dark phase, and is considered the main endocrinal signal for photoperiod duration (Simonneaux and Ribelayga, 2003). MLT is synthesized from serotonin (5-hydroxytryptamine, 5-HT) by a mechanism that involves the sequential activity of two enzymes, N-acetyltransferase (NAT) and hydroxyindole-O-methyltransferase (HIOMT), within a pathway that includes N-acetyl-serotonin as an intermediary product. Most physiological MLT effects result from the activation of two high-affinity G-protein coupled receptors, named MT₁ and MT₂, which are widely expressed in the mammalian central nervous system (Dubocovich et al., 2010). In addition to these high-affinity MLT receptors (with Kd in the pico-molar range), another low-affinity MLT binding site, termed MT₃, has been characterized as a melatonin-sensitive form of the human enzyme quinone reductase II (Nosjean et al., 2000). Melatonin acts also through non G protein-coupled receptors such as the retinoid orphan nuclear hormone receptors, referred to as RZR-alpha and RZR-beta at concentrations in the low nanomolar range. MLT also blocks calmodulin interactions with its target enzymes through induction of calmodulin phosphorylation by PKC-alpha (Paz Romero, 1998).

MLT has been involved in the control of many physiological circadian and seasonal functions such as sleep and reproduction, and some preclinical and clinical studies suggest a potential use of MLT in the management of pain.

In different rodent models of acute pain, inflammatory pain and neuropathic pain melatonin has potent antinociceptive effects (Ambriz-Tututi et al., 2009; Pang et al., 2001; Ambriz-Tututi and Granados-Soto, 2007).

mRNA expression of MT₁ and MT₂ receptors has been found in the mammalian spinal cord and MLT specific binding sites have been reported in the central gray substance (lamina X) which is a crucial region for peripheral pain control (Wan et al., 1996; Zahn et al., 2003).

In humans, the analgesic effects of melatonin on chronic pain have not been studied extensively and results are still very contradictory. Melatonin has been investigated mostly in fibromyalgia (FM), Irritable Bowel syndrome (IBS) and migraine.

FM includes tenderness, altered sleep pattern and a number of painful trigger points (American College of Rheumatology). One randomized double-blinded placebo controlled study has been carried out in FM (Hussein et al., 2011) using relatively low doses of MLT (5 mg), showing that MLT decreases pain and depression, and increase sleep.

IBS is a painful condition of the gastrointestinal system. This syndrome is classified according to Rome 2 criteria including abdominal pain, flatulence, constipation or diarrhoea and sleep disturbance. Three randomized double-blinded clinical trials have been conducted demonstrating an alleviation of pain (for review see Wilhelmsen et al., 2011).

Migraine is a common condition characterized by attacks of severe headache, neurologic dysfunction, sleep disturbances and pain-free intervals. A correlation between MLT levels and acute migraine has been found. One randomized double blind study was carried out in 32 patients with migraine and it was found that 3 mg of MLT can prevent migraine attacks, even if a recent study carried out with Circadin 2 mg failed to replicated these results (for review see Wilhelmsen et al, 2011).

MLT has also been demonstrated to be a potentiating agent of anesthetic drugs (Jarrat, 2011).

The specification refers to a number of documents, the contents of which are herein incorporated by reference in their entirety.

### SUMMARY

The present disclosure relates to methods and uses of N-(substituted-anilinoalkyl)acylamine melatonin ligands described herein and/or disclosed in PCT/CA2007/000055, herein incorporated by reference, for alleviating or treating pain and/or painful disorders. The present disclosure also relates to therapeutic compositions comprising such N-(substituted-anilinoalkyl)acylamine melatonin ligands or pharmaceutically acceptable salts thereof for the alleviation or treatment of pain and treatment of painful disorders.

In an aspect, the present disclosure includes a method of treating pain comprising administering to a subject in need thereof a compound of Formula I:

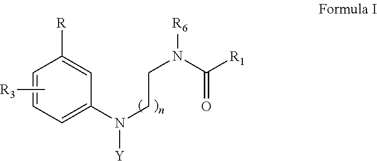

or a pharmaceutically acceptable salt thereof, wherein:
n is 1 or 2;
m is 0, 1 or 2;
p is 0, 1, 2, 3, 4, 5, 6, 7 or 8;
v is 2 or 3;
A is aryl or heteroaryl;
Z is O, S or NR₈;
Y is chosen from hydrogen, aryl, heteroaryl, C₁-C₆ alkyl, C₃-C₆ cycloalkyl, and

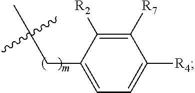

R is chosen from hydrogen, hydroxyl, —OCF₃, CF₃, C₁-C₈ alkyl, C₁-C₈ alkyloxy, C₁⁻C₈ alkylthio, halogen and —Z—(CH₂)ₚ-A;
R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl;
R₂ is chosen from hydrogen, C₁-C₄ alkyl, C₁-C₄ alkyloxy, OCF₃, CF₃, hydroxyl, and halogen;
R₃ is chosen from hydrogen, C₁-C₄ alkyl, C₁-C₄ alkyloxy, OCF₃, CF₃, hydroxyl and halogen;
R and R₃ may be connected together to form an —O—(CH₂)ᵥ bridge representing with the carbon atoms to which they are attached a 5- or 6-membered heterocyclic ring system;
R₄ is chosen from hydrogen, C₁-C₄ alkyl, C₁-C₄ alkyloxy, OCF₃, CF₃, hydroxyl, and halogen;
R₆ is chosen from hydrogen and C₁-C₆ alkyl;
R₇ is chosen from hydrogen, C₁-C₄ alkyl, C₁-C₄ alkyloxy, OCF₃, CF₃, hydroxyl and halogen;
R₈ is chosen from hydrogen and C₁-C₄ alkyl.

In an aspect, the present disclosure includes a method of alleviating pain comprising administering to a subject in need thereof a compound of Formula I herein disclosed.

In an aspect, the present disclosure includes a method of treating pain comprising administering to a subject in need thereof a therapeutically effective composition comprising one or more pharmaceutically acceptable excipients and a compound of Formula I herein disclosed or a pharmaceutically acceptable salt thereof.

In an aspect, the present disclosure includes a method of alleviating pain comprising administering to a subject in need thereof a therapeutically effective composition comprising one or more pharmaceutically acceptable excipients and a compound of Formula I herein disclosed or a pharmaceutically acceptable salt thereof.

In an aspect, the present disclosure includes the use of a pharmaceutically acceptable excipient and a compound of Formula I or a pharmaceutically acceptable salt thereof for treating pain.

In an aspect, the present disclosure includes the use of a pharmaceutically acceptable excipient and a compound of Formula I or a pharmaceutically acceptable salt thereof for alleviating pain.

In an aspect, the present disclosure includes a method of interacting with at least one of MT₁ and MT₂ MLT receptor subtypes for treating pain comprising administering to a subject in need thereof an effective amount of a compound of Formula I or a pharmaceutically acceptable salt thereof.

In an aspect, the present disclosure includes a method of interacting with at least one of MT₁ and MT₂ MLT receptor subtypes for alleviating pain comprising administering to a subject in need thereof an effective amount of a compound of Formula I or a pharmaceutically acceptable salt thereof.

In an aspect, the present disclosure relates to a compound of Formula:

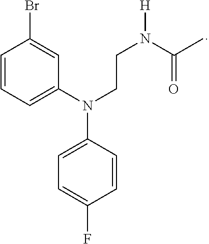 [SMILES: CC(=O)NCCN(c1ccc(F)cc1)c1cccc(Br)c1]

In an aspect, the present disclosure includes a therapeutically effective composition for treating a condition mediated by at least one of MT₁ and MT₂ receptors, comprising one or more pharmaceutically acceptable excipients and a compound of Formula:

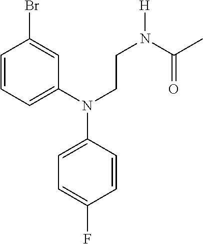 [SMILES: CC(=O)NCCN(c1ccc(F)cc1)c1cccc(Br)c1]

In an aspect, the present disclosure includes a method of interacting with at least one of MT₁ and MT₂ MLT receptor subtypes, comprising administering to a subject in need thereof an effective amount of a compound of Formula:

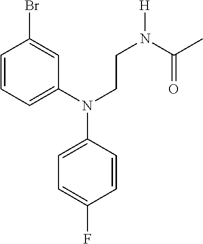 [SMILES: CC(=O)NCCN(c1ccc(F)cc1)c1cccc(Br)c1]

Other objects, features and advantages of the present disclosure will become apparent from the following detailed description. It should be understood, however, that the detailed description and the specific examples, while indicating illustrative embodiments are given by way of illustration only, since various changes and modifications within the spirit and scope of the disclosure will become apparent to those skilled in the art from this detailed description.

### BRIEF DESCRIPTION OF THE DRAWINGS

Having thus generally described the disclosure, reference will now be made to the accompanying drawings, showing by way of illustration a preferred embodiment thereof, and in which:

FIG. 1 shows a dose response of 5b (FIG. 1A) and 5p (FIG. 1B) during the hot plate test. All doses were administered 30 min prior to the test. 5b and 5p at 20 mg/kg increased the withdrawal temperature in comparison with vehicle (VEH) at 1 and 4 hours post-treatment. Data are expressed as Mean±SEM; **p<0.01, ***p<0.001 vs vehicle; ##p<0.01, ###p<0.001 vs 5b 10 mg/kg, two-way ANOVA, followed by SNK post-hoc test.

FIG. 2 shows the effects of MLT (150 mg/kg, s.c.), 5b (FIG. 2A) and 5p (FIG. 2B) (20 mg/kg, s.c.) and Acetaminophen (ACE) (200 mg/kg, s.c.) during the hot plate test. The drugs were injected 30 min prior to the test. MLT, 5b, 5p and ACE increased the withdrawal temperature in comparison with vehicle at 1 and 4 hours post-treatment. Data are expressed as Mean±SEM. *p<0.05, ***p<0.001 vs vehicle; ##p<0.01, ###p<0.001 vs 5b 20 mg/kg and ACE 200 mg/kg; two-way ANOVA, followed by SNK post-hoc test.

FIG. 3 shows a dose response of 5b (FIG. 3A) and 5p (FIG. 3B) in the formalin test. All doses were administered 30 min prior to the test. 5b and 5p at 20 and 40 mg/kg decreased the licking time during the phase 1, phase 2 and the total time of the formalin test in comparison with vehicle. Data are expressed as Mean±SEM; 6 rats per group. *p<0.05, **p<0.01, ***p<0.001 vs vehicle; ##p<0.01, ###p<0.001 vs 5b 10 mg/kg, one-way ANOVA, followed by SNK post-hoc test.

FIG. 4 shows the effects of MLT (150 mg/kg, s.c.), 5b (FIG. 4A) or 5p (FIG. 4B) (20 mg/kg, s.c.) and Ketoprofen (KET, 3 mg/kg, s.c.) in the formalin test. The drugs were injected 30 min prior to the test. MLT, 5b, 5p and KET decreased the licking time in comparison with vehicle at during the first phase, second phase and total time of the formalin test. Data are expressed as Mean±SEM; 6 rats per group. *p<0.05, ***p<0.001 vs vehicle; ##p<0.01, ###p<0.001 vs 5b 20 mg/kg and ACE 200 mg/kg; one-way ANOVA, followed by SNK post-hoc test.

FIG. 5 shows that MT2 partial agonist 5b reduced allodynia in a MT2 selective manner, with an efficacy comparable to gabapentin in a L5-L6 ligation model. (FIG. 5A) Time course of paw withdrawal threshold after Von Frey filaments stimulation in rats with L5-L6 spinal nerves ligation before (time 0) after (0.5-8 hours) subcutaneous administration of increasing doses of 5b (5, 10, 20 and 40 mg/kg); (FIG. 5B) Doses of 5b (20 mg/kg), gabapentin (GBP, 100 mg/kg), melatonin (MLT, 150 mg/kg), MT₂ antagonist 4P-PDOT (10 mg/kg) administered 10 min prior to 5b (20 mg/kg), in comparison with vehicle (VEH) treated rats. Intermittent line on the bottom of FIG. 5A and FIG. 5B represents the threshold cut-off (4 g) for allodynia in nerve ligated rats and values above this line are considered antiallodynic effective. Intermittent line on the top in FIG. 5A and FIG. 5B represents the threshold cut-off for sham-operated rats. (FIG. 5C) Area under the curve (AUC) of the antiallodynic effect (expressed in grams) of increasing doses of 5b compared to GBP (100 mg/kg), MLT (100 mg/kg). VEH (70% dimethylsulfoxide (DMSO) and 30% saline). Data are expressed as mean±SEM, (Animals per group=6). *p<0.05, **p<0.01 and ***p<0.001 vs. vehicle. ##p<0.01 and ###p<0.001 vs. melatonin by Bonferroni post-hoc test.

FIG. 6 shows that MT2 receptor partial agonist 5p reduced allodynia in the L5-L6 ligation model like GBP. Time course of paw withdrawal threshold after Von Frey filaments stimulation in rats with L5-L6 spinal nerves ligation before (time 0) and after (0.5-8 hours) (FIG. 6A) of increasing doses of 5p (5, 10, 20 and 40 mg/kg, s.c.); (FIG. 6B) of 5p (20 mg/kg), GBP (100 mg/kg), MLT (150 mg/kg), 4P-PDOT (10 mg/kg) administered 10 min before 5p (20 mg/kg), in comparison with vehicle (VEH) treated rats. Intermittent line on the bottom in A and B represents the threshold cut-off (4 g) for allodynia in nerve ligated rats and values above this line are considered antiallodynic effective. Intermittent line on the top in A and B represents the mean for sham-operated rats. (FIG. 6C) Area under the curve (AUC) of the antiallodynic effect (expressed in grams) of increasing doses of 5p compared to GBP (100 mg/kg), MLT (150 mg/kg). Data are expressed as mean±SEM, (Animals per group=6). *p<0.05, **p<0.01 and ***p<0.001 vs. VEH; #p<0.05, ###p<0.001 vs. MLT by Bonferroni post-hoc test.

FIG. 7 shows antihyperalgesic effects of MT2 partial agonists in the Spared Nerve Injury (SNI) model. (FIG. 7A) Effects of subcutaneous administration of 5b (10, 20 and 40 mg/kg), (FIG. 7B) 5p (10, 20 and 40 mg/kg), (FIG. 7C) MLT (150 mg/kg), gabapentin (GBP, 100 mg/kg), compared to 5b (20 mg/kg, sc) and 5p (20 mg/kg, sc) on mechanical withdrawal threshold in SNI rats. Each point represents threshold mean±SEM, (Animals per group=7-8). *p<0.05, **p<0.01 and ***p<0.001 indicate statistically significant difference vs. VEH. ⁺p<0.05, ⁺⁺p<0.01 ⁺⁺⁺p<0.01 indicate statistically significant difference between different doses of 5b and 5b. ##p<0.01, ###p<0.001 indicate statistically significant difference versus melatonin.

### DETAILED DESCRIPTION OF ILLUSTRATIVE EMBODIMENTS

In order to provide a clear and consistent understanding of the terms used in the present specification, a number of definitions are provided below. Moreover, the present description refers to a number of routinely used chemical terms; definitions of selected terms are provided for clarity and consistency.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or method steps.

The terms of degree such as “about”, “substantially” and “approximately” as used herein mean a reasonable amount of deviation of the modified term such that the end result is not significantly changed. Theses terms of degree should be construed as including a deviation of ±10% of the modified term if this deviation would not negate the meaning of the word it modifies.

The term “C₁-C₈ alkyl”, as used herein, is understood as being straight chain or branched chain alkyl groups non-limiting examples of which include methyl, ethyl, propyl, isopropyl, n-butyl, isobutyl, t-butyl, amyl, hexyl, heptyl and octyl.

The term “C₁-C₈ alkyloxy”, as used herein, is understood as being straight chain or branched chain alkyloxy groups, non-limiting examples of which include methoxy, ethoxy, propoxy, isopropoxy, n-butoxy, isobutoxy and t-butoxy.

The term “halogen”, as used herein, is understood as including fluorine, chlorine, bromine and iodine.

The term “C₃-C₆ cycloalkyl”, as used herein, is understood as being a carbon-based ring system, non-limiting examples of which include cyclopropyl, cyclobutyl, cyclopentyl and cyclohexyl.

The term “aryl”, as used herein, is understood as being an aromatic substituent which is a single ring or multiple rings fused together and which may optionally be substituted. When formed of multiple rings, at least one of the constituent rings is aromatic. In an embodiment, aryl substituents include phenyl, and naphthyl groups.

The term “heteroaryl”, as used herein, is understood as being unsaturated rings of five or six atoms containing one or two O- and/or S-atoms and/or one to four N-atoms, provided that the total number of hetero-atoms in the ring is 4 or less. The heteroaryl ring is attached by way of an available carbon or nitrogen atom. Non-limiting examples of heteroaryl groups include 2-, 3-, or 4-pyridyl, 4-imidazolyl, 4-thiazolyl, 2- and 3-thienyl, and 2- and 3-furyl. The term “heteroaryl”, as used herein, is understood as also including bicyclic rings wherein the five or six membered ring containing O, S and N-atoms as defined above is fused to a benzene or pyridyl ring. Non-limiting examples of bicyclic rings include but are not limited to 2- and 3-indolyl as well as 4- and 5-quinolinyl.

The term “heteroatom”, as used herein, is understood as being oxygen, sulfur or nitrogen.

The term “patient” or “subject”, as used herein, is understood as being any individual treated with the melatonin ligands of the present disclosure for the alleviation or treatment of pain. Patients include humans, as well as other animals such as companion animals, livestock and rodents such as for example rats. Patients may be afflicted by a painful condition or painful disorder or may be free of such a condition (i.e. treatment may be prophylactic, for example prior to surgery).

Administration and uses of solvates of the melatonin ligands of the present disclosure are also contemplated herein. Solvates of the compounds of Formula I are preferably hydrates.

The term “derivative” as used herein, is understood as being a substance which comprises the same basic carbon skeleton and carbon functionality in its structure as a given compound, but can also bear one or more substituents or rings.

The term “analogue” as used herein, is understood as being a substance similar in structure to another compound but differing in some slight structural detail.

The term “antagonist” as used herein, is understood as being any molecule that blocks, inhibits, or neutralizes a biological activity of the high affinity MLT receptors subtypes MT₂ and/or MT₁. In a similar manner, the term “agonist” as used herein, is understood as being any molecule that mimics a biological activity of native MLT. The term “partial agonist” as used herein, is understood as being any molecule that mimics the activity of endogenous MLT but is unable to achieve the maximal activity of MLT.

The term “salt(s)” as used herein, is understood as being acidic and/or basic salts formed with inorganic and/or organic acids or bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps.

As used herein “acute pain” refers to pain that is a normal predicted physiological response to a chemical, thermal or mechanical stimulus associated for example with surgery, trauma and/or acute illness which can be severe and is time limited. It usually decreases over a period of few minutes, hours, days or weeks.

As used herein “chronic pain” refers to pain that is persistent or repeated over an extended period of time. Chronic pain is pain associated with a chronic medical condition or extending beyond the period of tissue injury and normal healing. Chronic pain is variously defined as pain lasting more than a month, more than three months or more than six months. Chronic pain may be due to cancer (usually referred to as cancer pain), injury to nerves (usually referred to as neuropathic pain) or conditions other than cancer or neuropathy (usually referred to as chronic non-cancer pain, chronic non-malignant pain, or simply chronic pain). For example relating to a long term and/or incurable condition or disease, such as arthritis, irritable bowel syndrome and migraines. Chronic pain can be mild to severe and includes for example neuropathic pain.

Examples of acid addition salts include but are not limited to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, phosphoric, 2-hydroxyethanesulfonate, lactate, maleate, mandelate, methanesulfonate, 2-naphthalenesulfonate, nicotinate, oxalate, pectinate, persulfate, 3-phenylpropionate, picrate, pivalate, propionate, succinate, tartrate, thiocyanate, tosylate, and undecanoate.

Examples of base addition salts include but are not limited to alkali metal salts and alkaline earth metal salts. Non limiting examples of alkali metal salts include lithium, sodium and potassium salts. Non-limiting examples of alkaline earth metal salts include magnesium and calcium salts.

It is contemplated that any embodiment discussed in this specification can be implemented with respect to any method or composition of the disclosure, and vice versa. Furthermore, compositions of the disclosure can be used to achieve methods and uses of the disclosure.

The N-(substituted-anilinoalkyl)acylamines are a new class of MLT ligands disclosed in PCT/CA2007/000055, herein incorporated by reference. Compounds of Formula I have been demonstrated herein to have anesthetic-like properties. For example, it was found that during stereotaxical surgery, anesthetized animals administered with an intravenous dose of a N-(substituted-anilinoalkyl)acylamine required less anesthetic (chloral hydrate) to maintain deep anaesthesia. The pharmacological profile of several hypnotic drugs also includes anesthetic, anxiolytic and/or analgesic properties. Compounds of Formula I were tested and found to alleviate acute pain, inflammatory pain and neuropathic pain, as described further below.

In an aspect, the present disclosure includes methods and uses of melatonin ligands and pharmaceutically acceptable salts thereof having analgesic properties. More specifically, the present disclosure relates to methods and uses of (N-(substituted-anilinoalkyl)acylamines and pharmaceutically acceptable salts thereof having high binding affinity for the MT₂ and/or MT₁ melatonin receptors.

Accordingly, an aspect relates to a method of treating and/or alleviating pain comprising administering to a subject in need thereof a compound of Formula I:

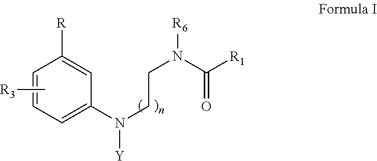

or a pharmaceutically acceptable salt thereof, wherein:
n is 1 or 2;
m is 0, 1 or 2;
p is 0, 1, 2, 3, 4, 5, 6, 7 or 8;
v is 2 or 3;
A is aryl or heteroaryl;
Z is O, S or NR₈;
Y is chosen from hydrogen, aryl, heteroaryl, C₁-C₆ alkyl, C₃-C₆ cycloalkyl, and

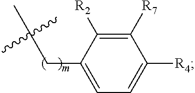

R is chosen from hydrogen, hydroxyl, —OCF₃, CF₃, C₁-C₈ alkyl, C₁-C₈ alkyloxy, C₁-C₈ alkylthio, halogen and —Z—(CH₂)ₚ-A;
R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl;
R₂ is chosen from hydrogen, C₁-C₄ alkyl, C₁-C₄ alkyloxy, OCF₃, CF₃, hydroxyl, and halogen;
R₃ is chosen from hydrogen, C₁-C₄ alkyl, C₁-C₄ alkyloxy, OCF₃, CF₃, hydroxyl and halogen;
R and R₃ may be connected together to form an —O—(CH₂)ᵥ bridge representing with the carbon atoms to which they are attached a 5- or 6-membered heterocyclic ring system;
R₄ is chosen from hydrogen, C₁-C₄ alkyl, C₁-C₄ alkyloxy, OCF₃, CF₃, hydroxyl, and halogen;
R₆ is chosen from hydrogen and C₁-C₆ alkyl;
R₇ is chosen from hydrogen, C₁-C₄ alkyl, C₁-C₄ alkyloxy, OCF₃, CF₃, hydroxyl and halogen;
R₈ is chosen from hydrogen and C₁-C₄ alkyl.

In an embodiment, n is 1 or 2; m is 0, or 1; p is 0, 1, 2, 3, or 4; A is phenyl; Z is O; Y is chosen from hydrogen, methyl, β-naphthyl, thiophene-3-yl, and

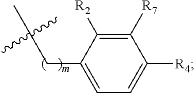

R is chosen from hydrogen, methoxy, Br and —Z—(CH₂)ₚ-A; R₁ is chosen from methyl, propyl and cyclobutyl; R₂ is hydrogen; R₃ is chosen from hydrogen, halogen and methoxy; R₄ is hydrogen or halogen; R₆ is hydrogen or methyl; R₇ is hydrogen, hydroxy or methoxy.

In another embodiment, R is H or methoxy; R₁ is methyl, ethyl, propyl, cyclopropyl, cyclobutyl or NHR₅, wherein R₅ is ethyl, H or propyl.

In yet another embodiment, the method of treating and/or alleviating pain comprises administering to a subject in need thereof a compound chosen from N-[2-(diphenylamino)ethyl]acetamide (5a), N-{2-[(3-Methoxyphenyl)-phenylamino]ethyl}acetamide (5b), N-[2-(bis-3-methoxyphenylamino)ethyl]acetamide (5c), N-{2-[(4-Methoxyphenyl)-3-methoxyphenylamino]ethyl}acetamide (5d), N-{2-[(4-Methoxyphenyl)-phenylamino]ethyl}acetamide (5e), N-{2-[(3-bromophenyl)-phenylamino]ethyl}acetamide (5f), N-{2-[(3-Methoxyphenyl)-β-naphthylamino]ethyl}acetamide (5g), N-{2-[(3-methoxyphenyl)(thiophen-2-yl)amino]ethyl}acetamide (5h), N-{2-[(3-pheny/butoxyphenyl)-phenyl-amino]ethyl}acetamide (5i), N-{2-[(3-Methoxyphenyl)-methylamino]ethyl}acetamide (5j), N-{2-[(3-Methoxyphenyl)-benzylamino]ethyl}acetamide (5k), N-{2-[(3-Methoxyphenyl)-amino]ethyl}acetamide (5l), N-{3-[(3-Methoxyphenyl)-methylamino]propyl}acetamide (5m), N-{2-[(3-Methoxyphenyl)-phenylamino]ethyl}butanamide (5n), N-{2-[(3-Methoxyphenyl)-phenylamino]ethyl}cyclobutancarboxamide (5o), N-{2-[(3-Bromophenyl)-4-fluorophenylamino]ethyl}acetamide (5p), N-Methyl-N-{2-[(3-methoxyphenyl)-phenylamino]ethyl}acetamide (6), N-{2[(3-butoxyphenyl)-methylamino]ethyl}acetamide, N-{2-[(3-hexyloxyphenyl)-methylamino]ethyl}acetamide, and N-{2-{[3-(4-phenylbutoxy)phenyl)-methylamino]}ethyl}acetamide.

In an embodiment, the method of treating and/or alleviating pain comprises administering to a subject in need thereof the compound N-{2-[(3-Methoxyphenyl)-phenylamino]ethyl}acetamide (5b). In another embodiment, the method of treating and/or alleviating pain comprises administering to a subject in need thereof the compound N-{2-[(3-Bromophenyl)-4-fluorophenylamino]ethyl}acetamide (5p).

In an embodiment, the pain to be treated and/or treated is chronic and/or acute pain. The acute pain can for example be acute tonic pain, or pain relating to surgery, e.g. post-surgical pain, surgical pain, and/or trauma.

In an embodiment, the pain is hyperalgesic pain or allodynic pain. In another embodiment, the pain is myalgic pain and/or inflammatory pain.

In another embodiment the pain is neuropathic pain and/or nociceptive pain. In a further embodiment, the nociceptive pain is visceral pain or somatic pain, for example musculo-skeletal pain or post-traumatic pain. In another embodiment, the neuropathic pain is peripheral neuropathic pain or central neuropathic pain.

In a further embodiment, the pain is back pain or joint pain. In yet another embodiment, the pain is head pain (e.g. headache)

In another embodiment, the pain is pain associated with a disorder or condition. In an embodiment, the disorder or condition is chosen from fibromyalgia, irritable bowel syndrome, arthritis, ulcer, diabetic neuropathy, sciatica and migraine. In another embodiment, the ulcer is a gastric ulcer. In another embodiment the pain associated to vulvodynia.

One or more types of pain can for example be treated and/or alleviated at the same time. One or more types of pain can for example be associated with a disorder or condition.

In an embodiment, the disclosure includes a method of alleviating or treating pain prophylactically comprising administering to a subject in need thereof a therapeutically effective amount of a compound of Formula I herein disclosed or a composition comprising one or more pharmaceutically acceptable excipients and a compound of Formula I herein disclosed. In an embodiment, the compound or composition is administered prior to surgery.

In an embodiment of the present disclosure, compounds of Formula I can be used to treat and/or alleviate pain. In one embodiment, the compounds of Formula I can induce an analgesic effect. In another embodiment, the compounds of Formula I can induce an antinociceptive effect. In a further embodiment, the compounds of Formula I can induce an antiallodynic effect. In yet another embodiment, the compounds of Formula I can induce an antihyperalgesic effect. In still a further embodiment, the compounds of Formula I can induce an anaesthetic effect.

An ordinary person of skill in the art would understand that the terms analgesic, antinociceptive, antiallodynic, antihyperalgesic and anaesthetic can be used interchangeably.

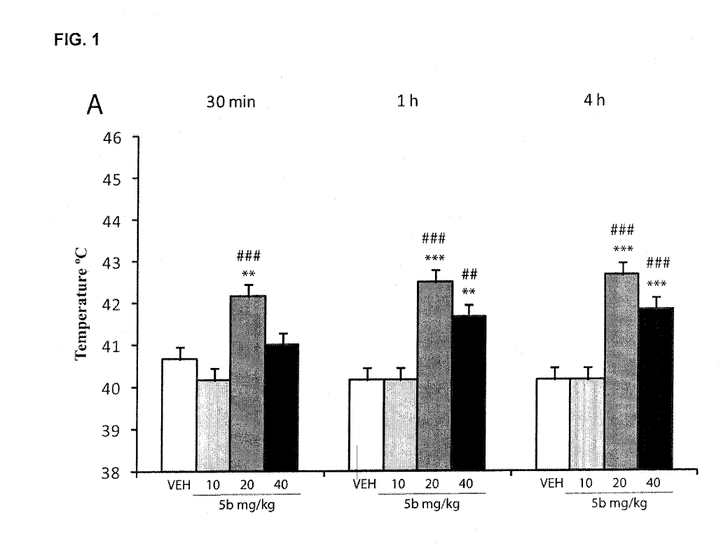

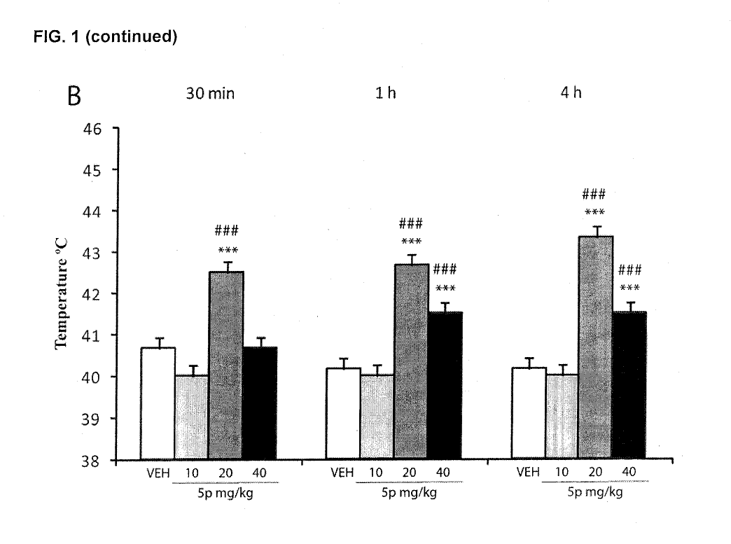

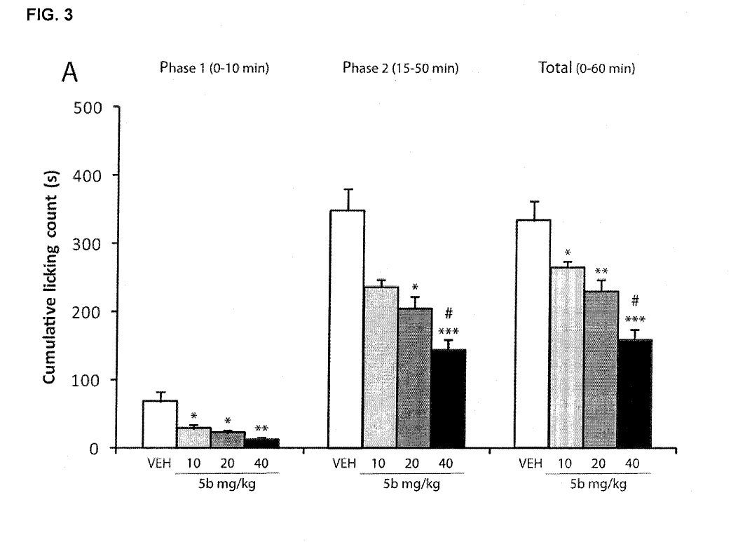

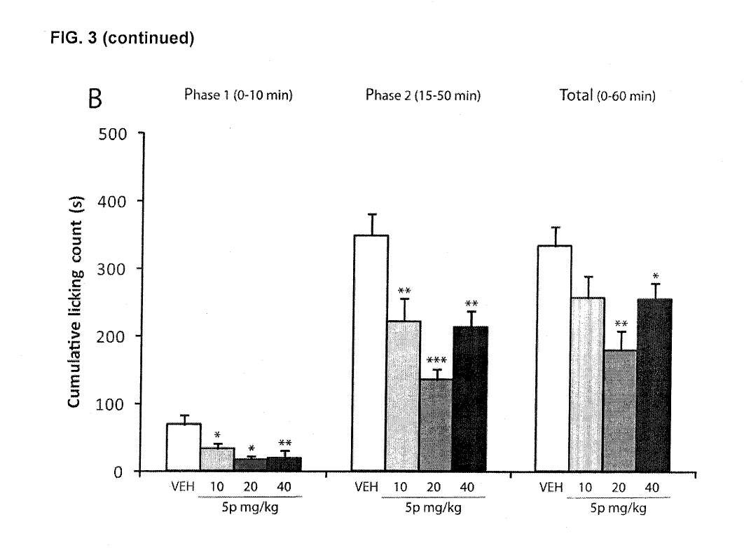

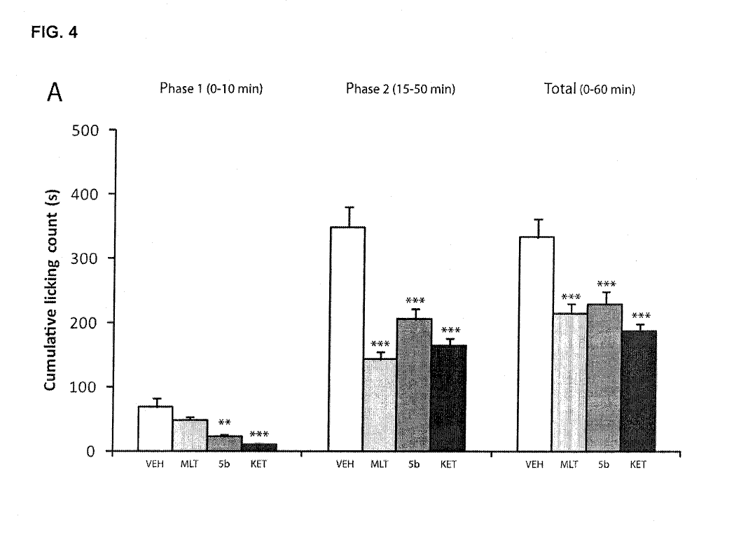

In another embodiment of the present disclosure, compounds of Formula I provide an antinociceptive effect and can be used to treat and/or alleviate pain. For example, in the hot plate test, the compounds 5b and 5p showed an antinociceptive effect on rats undergoing the hot plate test. There was a significant increase in withdrawal temperature in rats treated with compound 5b (FIG. 1A) or compound 5p (FIG. 1B) compared to untreated rats. The antinociceptive effect of compounds 5b and 5p were also demonstrated in the formalin test where licking time for rats treated with 5b or 5p was significantly decreased compared to untreated rats (FIG. 3).

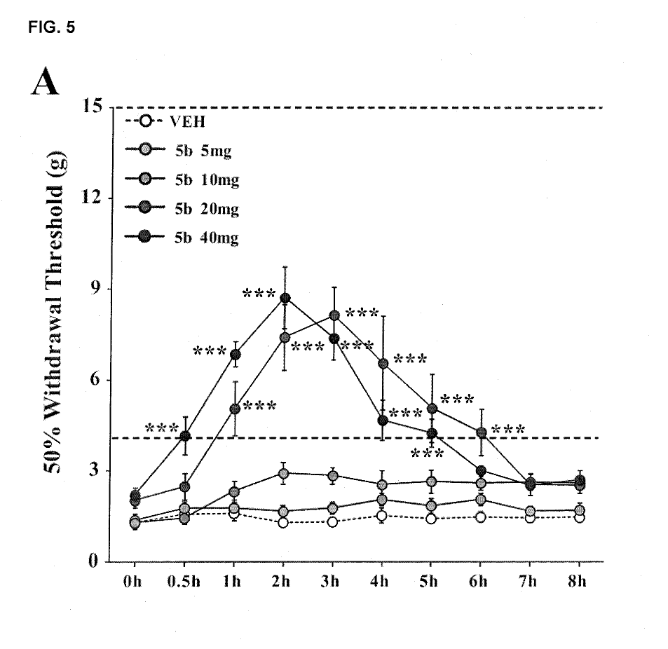

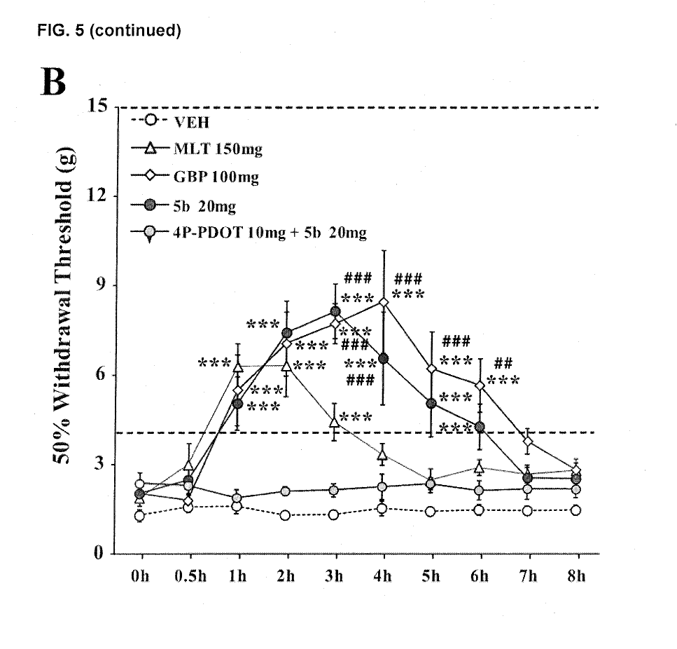

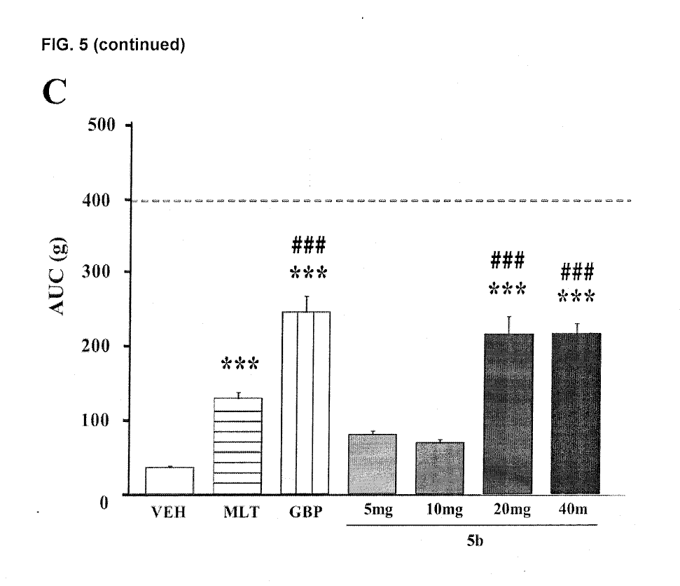

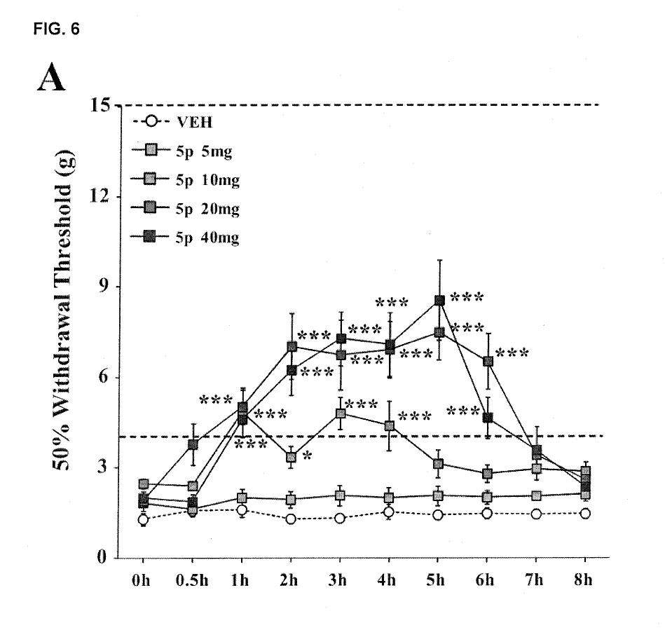

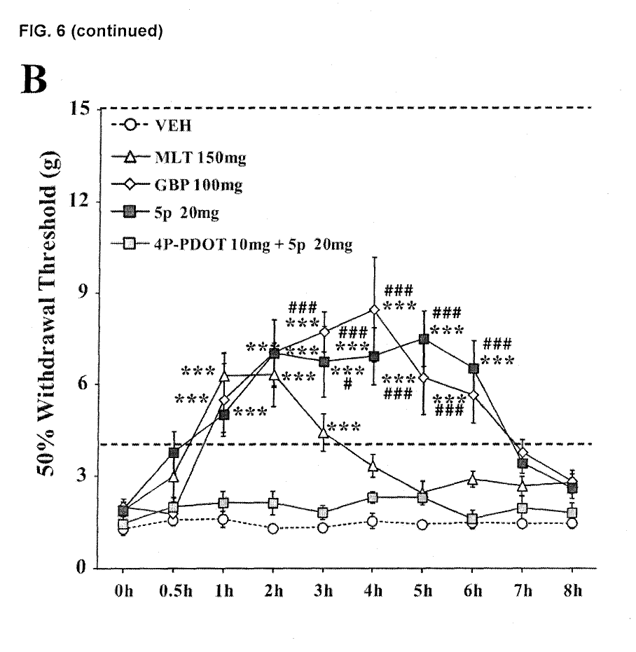

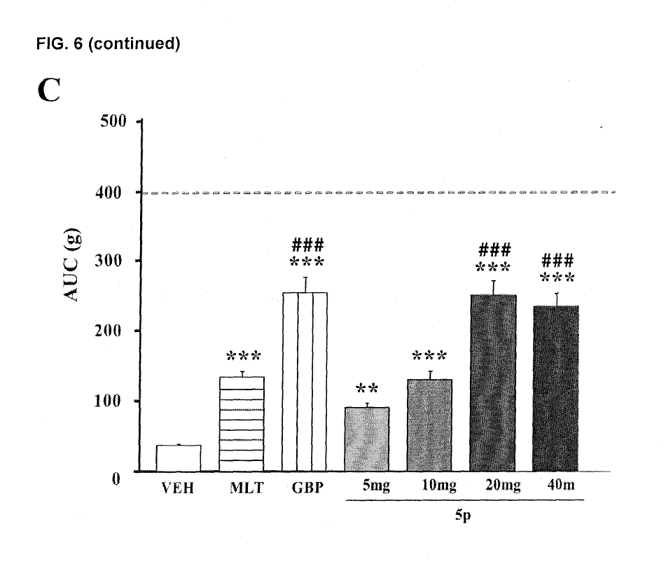

In another embodiment of the present disclosure, compounds of Formula I provide an antiallodynic effect and can be used to treat and/or alleviate pain. For example, compounds 5b and 5p are shown to have antiallodynic properties in rats. For example, administration of 5b or 5p compounds in allodynic rats (caused by L5-L6 spinal nerve ligation) reversed tactile allodynia as measured by an increase in the hindpaw withdrawal threshold to a mechanical stimulation compared to untreated allodynic rats (FIGS. 5 and 6).

In another embodiment of the present disclosure, compounds of Formula I provide an analgesic effect and can be used to treat and/or alleviate pain. For example, compounds 5b and 5p are shown to have analgesic properties in rats. For example, administration of 5b or 5p compounds in allodynic rats (caused by L5-L6 spinal nerve ligation) increased the analgesic effect as measured by an increase in the hindpaw withdrawal threshold to a mechanical stimulation compared to untreated allodynic rats (FIGS. 5 and 6).

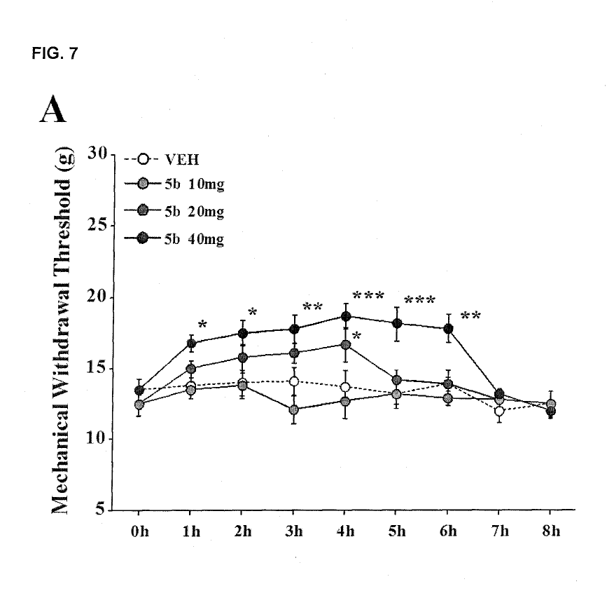

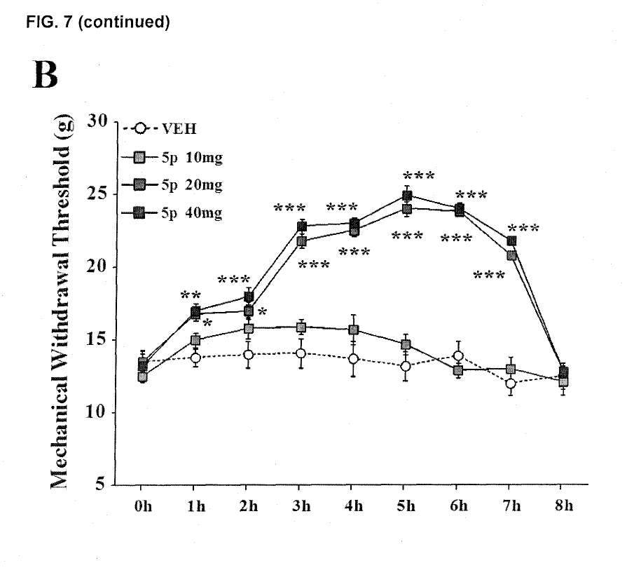

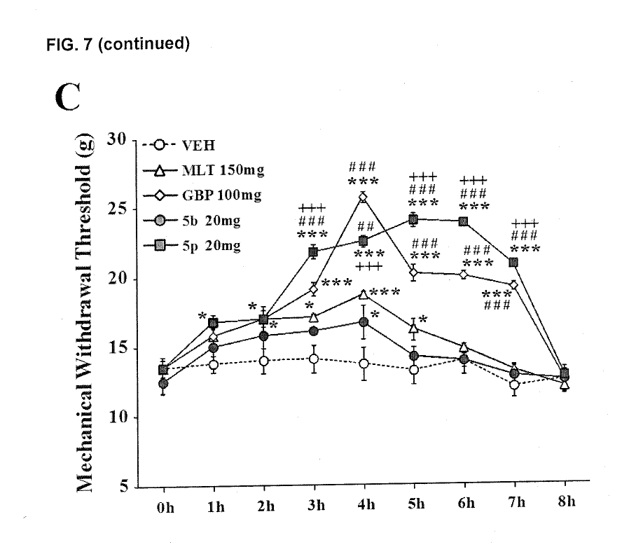

In yet another embodiment of the present disclosure, compounds of Formula I provide an antihyperalgesic effect. In an embodiment, compounds 5b and 5p are shown herein that they can be used to treat and/or alleviate pain. For example, rats with spared nerve injury treated with compounds 5b or 5p presented a significant decrease in mechanical withdrawal threshold compared to untreated rats (FIG. 7).

Another aspect of the present disclosure is a method of treating and/or alleviating pain comprising administering to a subject in need thereof a therapeutically effective composition comprising one or more pharmaceutically acceptable excipients and a compound of Formula I or a pharmaceutically acceptable salt thereof.

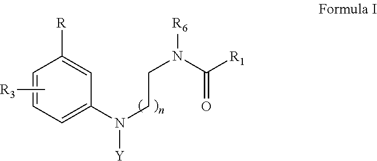

or a pharmaceutically acceptable salt thereof, wherein:
n is 1 or 2;
m is 0, 1 or 2;
p is 0, 1, 2, 3, 4, 5, 6, 7 or 8;
v is 2 or 3;
A is aryl or heteroaryl;
Z is O, S or NR₈;
Y is chosen from hydrogen, aryl, heteroaryl, C₁-C₆ alkyl, C₃-C₆ cycloalkyl, and

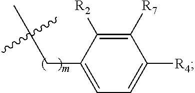

R is chosen from hydrogen, hydroxyl, —OCF₃, CF₃, C₁-C₈ alkyl, C₁-C₈ alkyloxy, C₁-C₈ alkylthio, halogen and —Z—(CH₂)ₚ-A;
R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl;
R₂ is chosen from hydrogen, C₁-C₄ alkyl, C₁-C₄ alkyloxy, OCF₃, CF₃, hydroxyl, and halogen;
R₃ is chosen from hydrogen, C₁-C₄ alkyl, C₁-C₄ alkyloxy, OCF₃, CF₃, hydroxyl and halogen;
R and R₃ may be connected together to form an —O—(CH₂)ᵥ bridge representing with the carbon atoms to which they are attached a 5- or 6-membered heterocyclic ring system;
R₄ is chosen from hydrogen, C₁-C₄ alkyl, C₁-C₄ alkyloxy, OCF₃, CF₃, hydroxyl, and halogen;
R₆ is chosen from hydrogen and C₁-C₆ alkyl;
R7 is chosen from hydrogen, C₁-C₄ alkyl, C₁-C₄ alkyloxy, OCF₃, CF₃, hydroxyl and halogen;
R₈ is chosen from hydrogen and C₁-C₄ alkyl.

In an embodiment, n is 1 or 2; m is 0, or 1; p is 0, 1, 2, 3, or 4; A is phenyl; Z is O; Y is chosen from hydrogen, methyl, β-naphthyl, thiophene-3-yl, and

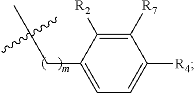

R is chosen from hydrogen, methoxy, Br and —Z—(CH₂)ₚ-A; R₁ is chosen from methyl, propyl and cyclobutyl; R₂ is hydrogen; R₃ is chosen from hydrogen, halogen and methoxy; R₄ is hydrogen or halogen; R₆ is hydrogen or methyl; R₇ is hydrogen, hydroxy or methoxy.

In another embodiment, R is H or methoxy; R₁ is methyl, ethyl, propyl, cyclopropyl, cyclobutyl or NHR₅, wherein R₅ is ethyl, H or propyl.

In yet another embodiment, the method of treating and/or alleviating pain comprises administering to a subject in need thereof a therapeutically effective composition comprising a compound chosen from N-[2-(diphenylamino)ethyl]acetamide (5a), N-{2-[(3-Methoxyphenyl)-phenylamino]ethyl}acetamide (5b), N-[2-(bis-3-methoxyphenylamino)ethyl]acetamide (5c), N-{2-[(4-Methoxyphenyl)-3-methoxyphenylamino]ethyl}acetamide (5d), N-{2-[(4-Methoxyphenyl)-phenylamino]ethyl}acetamide (5e), N-{2-[(3-bromophenyl)-phenylamino]ethyl}acetamide (5f), N-{2-[(3-Methoxyphenyl)-β-naphthylamino]ethyl}acetamide (5g), N-{2-[(3-methoxyphenyl)(thiophen-2-yl)amino]ethyl}acetamide (5h), N-{2-[(3-pheny/butoxyphenyl)-phenyl-amino]ethyl}acetamide (5i), N-{2-[(3-Methoxyphenyl)-methylamino]ethyl}acetamide (5j), N-{2-[(3-Methoxyphenyl)-benzylamino]ethyl}acetamide (5k), N-{2-[(3-Methoxyphenyl)-amino]ethyl}acetamide (5l), N-{3-[(3-Methoxyphenyl)-methylamino]propyl}acetamide (5m), N-{2-[(3-Methoxyphenyl)-phenylamino]ethyl}butanamide (5n), N-{2-[(3-Methoxyphenyl)-phenylamino]ethyl}cyclobutancarboxamide (5o), N-[2-[(3-Bromophenyl)-4-fluorophenylamino]ethyl}acetamide (5p), N-Methyl-N-{2-[(3-methoxyphenyl)-phenylamino]ethyl}acetamide (6), N-{2-[(3-butoxyphenyl)-methylamino]ethyl}acetamide, N-{2-[(3-hexyloxyphenyl)-methylamino]ethyl}acetamide, and N-{2-{[3-(4-phenylbutoxy)phenyl)-methylamino]}ethyl}acetamide.

In an embodiment, the method of treating and/or alleviating pain comprises administering to a subject in need thereof a therapeutically effective composition comprising the compound N-{2-[(3-Methoxyphenyl)-phenylamino]ethyl}acetamide (5b). In another embodiment, the method of treating and/or alleviating pain comprises administering to a subject in need thereof a therapeutically effective composition comprising the compound N-{2-[(3-Bromophenyl)-4-fluorophenylamino]ethyl}acetamide (5p).

In an embodiment, the pain to be treated and/or treated is chronic and/or acute pain. The acute pain can for example be acute tonic pain, or pain relating to surgery, e.g. post-surgical pain, surgical pain, and/or trauma. In an embodiment, the pain is hyperalgesic pain or allodynic pain. In another embodiment, the pain is myalgic pain and/or inflammatory pain. In another embodiment the pain is neuropathic pain and/or nociceptive pain. In a further embodiment, the nociceptive pain is visceral pain or somatic pain, for example musculo-skeletal pain or post-traumatic pain. In another embodiment, the neuropathic pain is peripheral neuropathic pain or central neuropathic pain. In a further embodiment, the pain is back pain or joint pain. In yet another embodiment, the pain is head pain (e.g. headache). In another embodiment, the pain is pain associated with a disorder or condition. In an embodiment, the disorder or condition is chosen from fibromyalgia, irritable bowel syndrome, arthritis, ulcer, diabetic neuropathy, sciatica and migraine. In another embodiment, the ulcer is a gastric ulcer. In another embodiment the pain associated to vulvodynia. One or more types of pain can for example be treated and/or alleviated at the same time. One or more types of pain can for example be associated with a disorder or condition.

A further aspect is a use of a pharmaceutical composition comprising a pharmaceutically acceptable excipient and a compound of Formula I or a pharmaceutically acceptable salt thereof for treating and/or alleviating pain:

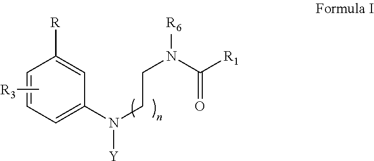

or a pharmaceutically acceptable salt thereof, wherein:
n is 1 or 2;
m is 0, 1 or 2;
p is 0, 1, 2, 3, 4, 5, 6, 7 or 8;
v is 2 or 3;
A is aryl or heteroaryl;
Z is O, S or NR₈;
Y is chosen from hydrogen, aryl, heteroaryl, C₁-C₆ alkyl, C₃-C₆ cycloalkyl, and

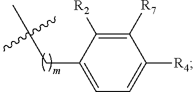

R is chosen from hydrogen, hydroxyl, —OCF₃, CF₃, C₁-C₈ alkyl, C₁-C₈ alkyloxy, C₁-C₈ alkylthio, halogen and —Z—(CH₂)ₚ-A;
R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl;
R₂ is chosen from hydrogen, C₁-C₄ alkyl, C₁-C₄ alkyloxy, OCF₃, CF₃, hydroxyl, and halogen;
R₃ is chosen from hydrogen, C₁-C₄ alkyl, C₁-C₄ alkyloxy, OCF₃, CF₃, hydroxyl and halogen;
R and R₃ may be connected together to form an —O—(CH₂)ᵥ bridge representing with the carbon atoms to which they are attached a 5- or 6-membered heterocyclic ring system;
R₄ is chosen from hydrogen, C₁-C₄ alkyl, C₁-C₄ alkyloxy, OCF₃, CF₃, hydroxyl, and halogen;
R₆ is chosen from hydrogen and C₁-C₆ alkyl;
R₇ is chosen from hydrogen, C₁-C₄ alkyl, C₁-C₄ alkyloxy, OCF₃, CF₃, hydroxyl and halogen;
R₈ is chosen from hydrogen and C₁-C₄ alkyl.

In an embodiment, n is 1 or 2; m is 0, or 1; p is 0, 1, 2, 3, or 4; A is phenyl; Z is O; Y is chosen from hydrogen, methyl, β-naphthyl, thiophene-3-yl, and

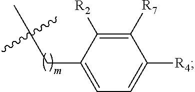

R is chosen from hydrogen, methoxy, Br and —Z—(CH₂)ₚ-A; R₁ is chosen from methyl, propyl and cyclobutyl; R₂ is hydrogen; R₃ is chosen from hydrogen, halogen and methoxy; R₄ is hydrogen or halogen; R₆ is hydrogen or methyl; R₇ is hydrogen, hydroxy or methoxy.

In another embodiment, R is H or methoxy; R₁ is methyl, ethyl, propyl, cyclopropyl, cyclobutyl or NHR₅, wherein R₅ is ethyl, H or propyl.

In an embodiment, the compound of Formula I disclosed herein and/or the pharmaceutical composition comprising a pharmaceutically acceptable excipient and a compound of Formula I disclosed herein used for treating and/or alleviating pain is chosen from N-[2-(diphenylamino)ethyl]acetamide (5a), N-{2-[(3-Methoxyphenyl)-phenylamino]ethyl}acetamide (5b), N-[2-(bis-3-methoxyphenylamino)ethyl]acetamide (5c), N-{2-[(4-Methoxyphenyl)-3-methoxyphenylamino]ethyl}acetamide (5d), N-{2-[(4-Methoxyphenyl)-phenylamino]ethyl}acetamide (5e), N-{2-[(3-bromophenyl)-phenylamino]ethyl}acetamide (5f), N-{2-[(3-Methoxyphenyl)-β-naphthylamino]ethyl}acetamide (5g), N-{2[(3-methoxyphenyl)(thiophen-2-yl)amino]ethyl}acetamide (5h), N-{2-[(3-pheny/butoxyphenyl)-phenyl-amino]ethyl}acetamide (5i), N-{2-[(3-Methoxyphenyl)-methylamino]ethyl}acetamide (5j), N-{2-[(3-Methoxyphenyl)-benzylamino]ethyl}acetamide (5k), N-{2-[(3-Methoxyphenyl)-amino]ethyl}acetamide (5l), N-{3-[(3-Methoxyphenyl)-methylamino]propyl}acetamide (5m), N-{2-[(3-Methoxyphenyl)-phenylamino]ethyl}butanamide (5n), N-{2-[(3-Methoxyphenyl)-phenylamino]ethyl}cyclobutancarboxamide (5o), N-{2-[(3-Bromophenyl)-4-fluorophenylamino]ethyl}acetamide (5p), N-Methyl-N-{2-[(3-methoxyphenyl)-phenylamino]ethyl}acetamide (6), N-{2-[(3-butoxyphenyl)-methylamino]ethyl}acetamide, N-{2-[(3-hexyloxyphenyl)-methylamino]ethyl}acetamide, and N-{2-{[3-(4-phenylbutoxy)phenyl)-methylamino]}ethyl}acetamide.

In an embodiment, the compound of Formula I disclosed herein and/or the pharmaceutical composition comprising a pharmaceutically acceptable excipient and a compound of Formula I disclosed herein used for treating and/or alleviating pain is N-{2-[(3-Methoxyphenyl)-phenylamino]ethyl}acetamide (5b). In another embodiment, the compound of Formula I disclosed herein and/or the pharmaceutical composition comprising a pharmaceutically acceptable excipient and a compound of Formula I disclosed herein used for treating and/or alleviating pain is N-{2-[(3-Bromophenyl)-4-fluorophenylamino]ethyl}acetamide (5p).

In an embodiment, the use is for treating and/or alleviating chronic and/or acute pain. In another embodiment, the use is for alleviating and/or treating pain chosen from myalgic pain, inflammatory pain, neuropathic pain and/or nociceptive pain. In another embodiment, the use is for alleviating and/or treating acute pain wherein the acute pain is post-surgical pain, acute tonic pain, and/or trauma pain. In yet another embodiment, the use is for alleviating and/or treating back pain, joint pain and/or head pain (e.g. headache). In an embodiment, the use is for alleviating and/or treating hyperalgesic pain or allodynic pain. In another embodiment, the use is for alleviating and/or treating myalgic pain and/or inflammatory pain. In another embodiment, the use is for alleviating and/or treating nociceptive pain chosen from visceral pain or somatic pain and/or alleviating or treating neuropathic pain chosen from peripheral neuropathic pain or central neuropathic pain. In yet another embodiment, the use is for alleviating and/or treating pain associated with a disorder or condition and the disorder or condition is chosen from fibromyalgia, irritable bowel syndrome, arthritis, ulcer, diabetic neuropathy, sciatica and migraine. In another embodiment, the ulcer is a gastric ulcer. In another embodiment the pain associated to vulvodynia. One or more types of pain can for example be treated and/or alleviated at the same time. One or more types of pain can for example be associated with a disorder or condition.

In an embodiment, the pharmaceutical composition comprises from about 0.1% to about 99% by weight of the compound of Formula I disclosed herein or a pharmaceutically acceptable salt thereof.

In another embodiment, the pharmaceutical composition comprises from about 10% to about 60% by weight of the compound of Formula I disclosed herein or a pharmaceutically acceptable salt thereof.

In an embodiment, the pharmaceutical composition comprises from about 20% to about 50% by weight of the compound of Formula I disclosed herein or a pharmaceutically acceptable salt thereof, 30% to about 40% by weight of the compound of Formula I disclosed herein or a pharmaceutically acceptable salt thereof, 43% to about 47% by weight of the compound of Formula I disclosed herein or a pharmaceutically acceptable salt thereof.

In another embodiment, the pharmaceutical composition comprises from about 0.1% to about 10% by weight of the compound of Formula I disclosed herein or a pharmaceutically acceptable salt thereof; about 10% to about 20% by weight of the compound of Formula I disclosed herein or a pharmaceutically acceptable salt thereof; about 20% to about 30% by weight of the compound of Forniula I disclosed herein or a pharmaceutically acceptable salt thereof; about 30% to about 40% by weight of the compound of Formula I disclosed herein or a pharmaceutically acceptable salt thereof; about 40% to about 50% by weight of the compound of Formula I disclosed herein or a pharmaceutically acceptable salt thereof; about 50% to about 60% by weight of the compound of Formula I disclosed herein or a pharmaceutically acceptable salt thereof; about 60% to about 70% by weight of the compound of Formula I disclosed herein or a pharmaceutically acceptable salt thereof; about 70% to about 80% by weight of the compound of Formula I disclosed herein or a pharmaceutically acceptable salt thereof; about 80% to about 90% by weight of the compound of Formula I disclosed herein or a pharmaceutically acceptable salt thereof; about 90% to about 99% by weight of the compound of Formula I disclosed herein or a pharmaceutically acceptable salt thereof.

In non-limiting embodiments, for example, the pharmaceutical composition may comprise about 10.0, about 11.0, about 12.0, about 13.0, about 14.0, about 15.0, about 16.0, about 17.0, about 18.0, about 19.0, about 20.0, about 21.0, about 22.0, about 23.0, about 24.0, about 25.0, about 26.0, about 27.0, about 28.0, about 29.0, about 30.0, about 31.0, about 32.0, about 33.0, about 34.0, about 35.0, about 36.0, about 37.0, about 38.0, about 39.0, about 40.0, about 41.0, about 42.0, about 43.0, about 44.0, about 45.0, about 46.0, about 47.0, about 48.0, about 49.0, about 50.0, about 51.0, about 52.0, about 53.0, about 54.0, about 55.0, about 56.0, about 57.0, about 58.0, about 59.0, about 60.0% by weight of the compound of Formula I disclosed herein or a pharmaceutically acceptable salt thereof.

In yet a further embodiment, the present disclosure relates to pharmaceutical compositions comprising a therapeutically effective amount of one or more of the melatonin ligands or pharmaceutically acceptable salts thereof as defined herein, and at least one pharmaceutically acceptable excipient, non-limiting examples of which are carriers and diluents, for use in alleviating and/or treating pain. The term “therapeutically effective amount” is understood as being an amount of melatonin ligand or pharmaceutically acceptable salts thereof as defined herein, required upon administration to a patient in order to for example, reduce pain intensity, and/or alleviate pain, and/or treat or inhibit pain associated with a painful condition or disease. Therapeutic methods comprise the step of treating patients in a pharmaceutically acceptable manner with the melatonin ligands or pharmaceutically acceptable salts thereof as disclosed herein, or with compositions comprising such melatonin ligands or pharmaceutically acceptable salts thereof. Such compositions may be in the form of tablets, coated tablets, capsules, caplets, powders, granules, lozenges, suppositories, reconstitutable powders, syrups, liquid preparations such as oral or sterile parenteral solutions, dispersions or suspensions including nasal sprays or drops, as well as injectable formulations and transdermal formulations.

In an embodiment, the transdermal formulations comprise a carrier chosen from an aqueous based cream, oil, gel base, ointment and patch.

The melatonin ligands or pharmaceutically acceptable salts thereof of the present disclosure may be administered alone or in combination with pharmaceutically acceptable carriers. The proportion of each carrier is determined by the solubility and chemical nature of the compound, the route of administration, and standard pharmaceutical practice. In order to ensure consistency of administration, in an embodiment of the present disclosure, the pharmaceutical composition is in the form of a unit dose. The unit dose presentation forms for oral administration may be tablets, coated tablets and capsules and may contain conventional excipients. Non-limiting examples of conventional excipients include binding agents such as acacia, gelatin, sorbitol, or polyvinylpyrrolidone; fillers such as lactose, dextrose, saccharose, sugar, maize-starch, calcium phosphate, sorbitol or glycine; tabletting lubricants such as talc, stearic acid, calcium or magnesium stearate, polyethylene glycols, gums, gels; disintegrants such as starch, polyvinylpyrrolidone, sodium starch glycollate or microcrystalline cellulose; mannitol, hydroxypropyl cellulose, sodium starch glycolate, hypromellose, titanium dioxide or pharmaceutically acceptable wetting agents such as sodium lauryl sulphate.

The melatonin ligands or pharmaceutically acceptable salts thereof of the present disclosure may be injected parenterally; this being intramuscularly, intravenously, or subcutaneously. For parenteral administration, the melatonin ligands or pharmaceutically acceptable salts thereof may be used in the form of sterile solutions containing solutes for example, sufficient saline or glucose to make the solution isotonic.

The melatonin ligands or pharmaceutically acceptable salts thereof of the present disclosure may also be administered via transdermal routes using dermal or skin patches.

The melatonin ligands or pharmaceutically acceptable salts thereof may be administered orally in the form of tablets, coated tablets, capsules, or granules, containing suitable excipients non-limiting examples of which are starch, lactose, white sugar and the like. The melatonin ligands or pharmaceutically acceptable salts thereof may be administered orally in the form of solutions which may contain coloring and/or flavoring agents. The melatonin ligands or pharmaceutically acceptable salts thereof may also be administered sublingually in the form of tracheas or lozenges in which the active ingredient(s) is/are mixed with sugar or corn syrups, flavoring agents and dyes, and then dehydrated sufficiently to make the mixture suitable for pressing into solid form.

Sublingual, buccal, transnasal, intratecal routes of administration are also contemplated.

The solid oral compositions may be prepared by conventional methods of blending, granulation, compression, coating, filling, tabletting, or the like. Repeated blending operations may be used to distribute the active agent throughout those compositions employing large quantities of fillers. Such operations are, of course, conventional in the art. The tablets may be coated according to methods well known in normal pharmaceutical practice, in particular with an enteric coating.

Oral liquid preparations may be in the form of emulsions, suspensions, syrups, or elixirs, or may be presented as a dry product for reconstitution with water or other suitable vehicle before use. Such liquid preparations may or may not contain conventional additives. Non limiting examples of conventional additives include suspending agents such as sorbitol, syrup, natural gums, agar, methyl cellulose, gelatin, pectin, sodium alginate, hydroxyethylcellulose, carboxymethylcellulose, aluminum stearate gel, or hydrogenated edible fats; emulsifying agents such as sorbitan monooleate or acaci; non-aqueous vehicles (which may include edible oils) such as almond oil, fractionated coconut oil, oily esters chosen from glycerine, propylene glycol, ethylene glycol, and ethyl alcohol; preservatives such as for instance methyl para-hydroxybenzoate, ethyl para-hydroxybenzoate, n-propyl parahydroxybenzoate, n-butyl parahydroxybenzoate or sorbic acid; and, if desired conventional flavoring such as saccharose, glycerol, mannitol, sorbitol, or coloring agents.

For parenteral administration, fluid unit dosage forms may be prepared by utilizing the melatonin ligands or pharmaceutically acceptable salts thereof and a sterile vehicle (i.e. sterile water), and, depending on the concentration employed, the melatonin ligands or pharmaceutically acceptable salts thereof may be either suspended or dissolved in the vehicle. Other suitable vehicles may include olive oil, ethyl oleate, and glycols. If needed, a suitable quantity of lidocaine hydrochloride may also be included. Once in solution, the melatonin ligands or pharmaceutically acceptable salts thereof may be injected and filter sterilized before filling a suitable vial or ampoule followed by subsequently sealing the carrier or storage package. Adjuvants, such as a local anesthetic, a preservative or a buffering agent, may be dissolved in the vehicle prior to use. Stability of the pharmaceutical composition may be enhanced by freezing the composition after filling the vial and removing the water under vacuum, (e.g., freeze drying). Parenteral suspensions may be prepared in substantially the same manner, except that the melatonin ligands or pharmaceutically acceptable salts thereof should be suspended in the vehicle rather than being dissolved, and, further, sterilization is not achievable by filtration. The melatonin ligands or pharmaceutically acceptable salts thereof may be sterilized, however, by exposing it to ethylene oxide before suspending it in the sterile vehicle. A surfactant or wetting solution may be advantageously included in the composition to facilitate uniform distribution of the melatonin ligands or pharmaceutically acceptable salts thereof.

The melatonin ligands or pharmaceutically acceptable salts thereof may be administered in the form of suppositories. Suppositories may contain pharmaceutically acceptable vehicles such as cocoa butter, polyethylene glycol, sorbitan, esters of fatty acids, lecithin and the like.

The pharmaceutical compositions of the present disclosure for alleviation and/or treatment of pain comprise a pharmaceutically effective amount of at least one melatonin ligand or pharmaceutically acceptable salt thereof as described herein and one or more pharmaceutically acceptable carriers, excipients or diluents. In an embodiment of the present disclosure, the pharmaceutical compositions contain from about 0.1% to about 99% by weight of a melatonin ligand or pharmaceutically acceptable salt thereof as disclosed herein. In a further embodiment of the present disclosure, the pharmaceutical compositions contain from about 10% to about 60% by weight of a melatonin ligand or pharmaceutically acceptable salt thereof as disclosed herein, depending on which method of administration is employed. Physicians will determine the most-suitable dosage of the present therapeutic agents (the melatonin ligands or pharmaceutically acceptable salts thereof). Dosages may vary with the mode of administration and the particular melatonin ligand chosen. In addition, the dosage may vary with the particular patient under treatment. The dosage of the melatonin ligand or pharmaceutically acceptable salt thereof used in the treatment may vary, depending on the degree of MLT activity, the relative efficacy of the compound and the judgment of the treating physician.

In a non-limiting embodiment, the MLT ligands of the present disclosure are suitable for oral administration.

In an embodiment of the present disclosure, the pharmaceutical compositions for alleviating and/or treating pain comprise a therapeutically effective amount of one or more of the melatonin ligands or pharmaceutically acceptable salts thereof as defined herein, and at least one pharmaceutically acceptable excipient, non-limiting examples of which are carriers and diluents.

In an aspect, the present disclosure includes a method of interacting with at least one of MT₁ and MT₂ MLT receptor subtypes for treating and/or alleviating pain comprising administering to a subject in need thereof an effective amount of a compound of Formula I or a pharmaceutically acceptable salt thereof:

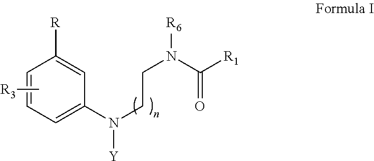

or a pharmaceutically acceptable salt thereof, wherein:
n is 1 or 2;
m is 0, 1 or 2;
p is 0, 1, 2, 3, 4, 5, 6, 7 or 8;
v is 2 or 3;
A is aryl or heteroaryl;
Z is O, S or NR₈;
Y is chosen from hydrogen, aryl, heteroaryl, C₁-C₆ alkyl, C₃-C₆ cycloalkyl, and

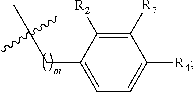

R is chosen from hydrogen, hydroxyl, —OCF₃, CF₃, C₁-C₈ alkyl, C₁-C₈ alkyloxy, C₁-C₈ alkylthio, halogen and —Z—(CH₂)ₚ-A;
R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl;
R₂ is chosen from hydrogen, C₁-C₄ alkyl, C₁-C₄ alkyloxy, OCF₃, CF₃, hydroxyl, and halogen;
R₃ is chosen from hydrogen, C₁-C₄ alkyl, C₁-C₄ alkyloxy, OCF₃, CF₃, hydroxyl and halogen;
R and R₃ may be connected together to form an —O—(CH₂)ᵥ bridge representing with the carbon atoms to which they are attached a 5- or 6-membered heterocyclic ring system;
R₄ is chosen from hydrogen, C₁-C₄ alkyl, C₁-C₄ alkyloxy, OCF₃, CF₃, hydroxyl, and halogen;
R₆ is chosen from hydrogen and C₁-C₆ alkyl;
R₇ is chosen from hydrogen, C₁-C₄ alkyl, C₁-C₄ alkyloxy, OCF₃, CF₃, hydroxyl and halogen;
R₈ is chosen from hydrogen and C₁-C₄ alkyl.

In an embodiment, the compound of Formula I is a ligand to MLT receptor subtypes MT₁ and/or MT₂. In another embodiment, the compound of Formula I is an agonist of MLT receptor subtypes MT₁ and/or MT₂. For example, the compound of Formula I can be a selective agonist for MLT receptor subtypes MT₁ and/or MT₂. For example, the compound of Formula I can be a partial agonist for MLT receptor subtypes MT₁ and/or MT₂. For example, the compound of Formula I can be an antagonist for MLT receptor subtypes MT₁ and/or MT₂.

For example, compounds 5b and 5p are ligands to MLT receptor subtypes MT₁ and/or MT₂. As shown in Table 1, the binding affinity and intrinsic activity of several compounds of Formula I was measured in CHO cells. Compound 5b has partial agonist activity for the MLT receptor subtypes MT₁ and MT₂. Compound 5i shows antagonist activity for the MLT receptor subtypes MT₁ and MT₂. Compound 5p shows antagonist for the receptor subtypes MT₁ and partial agonist activity for the receptor subtypes MT₂.

In an embodiment, n is 1 or 2; m is 0, or 1; p is 0, 1, 2, 3, or 4; A is phenyl; Z is O; Y is chosen from hydrogen, methyl, β-naphthyl, thiophene-3-yl, and

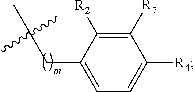

R is chosen from hydrogen, methoxy, Br and —Z—(CH₂)ₚ-A; R₁ is chosen from methyl, propyl and cyclobutyl; R₂ is hydrogen; R₃ is chosen from hydrogen, halogen and methoxy; R₄ is hydrogen or halogen; R₆ is hydrogen or methyl; R₇ is hydrogen, hydroxy or methoxy.

In another embodiment, R is H or methoxy; R₁ is methyl, ethyl, propyl, cyclopropyl, cyclobutyl or NHR₅, wherein R₅ is ethyl, H or propyl.

In yet another embodiment, the method of interacting with at least one of MT₁ and MT₂ MLT receptor subtypes for treating and/or alleviating pain comprises administering to a subject in need thereof an effective amount of a compound chosen from N-[2-(diphenylamino)ethyl]acetamide (5a), N-{2-[(3-Methoxyphenyl)-phenylamino]ethyl}acetamide (5b), N-[2-(bis-3-methoxyphenylamino)ethyl]acetamide (5c), N-{2-[(4-Methoxyphenyl)-3-methoxyphenylamino]ethyl}acetamide (5d), N-{2-[(4-Methoxyphenyl)-phenylamino]ethyl}acetamide (5e), N-{2-[(3-bromophenyl)-phenylamino]ethyl}acetamide (5f), N-{2-[(3-Methoxyphenyl)-β-naphthylamino]ethyl}acetamide (5g), N-{2-[(3-methoxyphenyl)(thiophen-2-yl)amino]ethyl}acetamide (5h), N-{2-[(3-pheny/butoxyphenyl)-phenyl-amino]ethyl}acetamide (5i), N-{2-[(3-Methoxyphenyl)-methylamino]ethyl}acetamide (5j), N-(2-[(3-Methoxyphenyl)-benzylamino]ethyl}acetamide (5k), N-{2-[(3-Methoxyphenyl)-amino]ethyl}acetamide (5l), N-{3-[(3-Methoxyphenyl)-methylamino]propyl}acetamide (5m), N-{2-[(3-Methoxyphenyl)-phenylamino]ethyl}butanamide (5n), N-{2-[(3-Methoxyphenyl)-phenylamino]ethyl}cyclobutancarboxamide (5o), N-{2-[(3-Bromophenyl)-4-fluorophenylamino]ethyl}acetamide (5p), N-Methyl-N-{2-[(3-methoxyphenyl)-phenylamino]ethyl}acetamide (6), N-{2-[(3-butoxyphenyl)-methylamino]ethyl}acetamide, N-{2-[(3-hexyloxyphenyl)-methylamino]ethyl}acetamide, and N-{2-{[3-(4-phenylbutoxy)phenyl)-methylamino]}ethyl}acetamide.

In an embodiment, the method of interacting with at least one of MT₁ and MT₂ MLT receptor subtypes for treating and/or alleviating pain comprises administering to a subject in need thereof an effective amount of the compound N-{2-[(3-Methoxyphenyl)-phenylamino]ethyl}acetamide (5b). In another embodiment, the method of interacting with at least one of MT₁ and MT₂ MLT receptor subtypes for treating and/or alleviating pain comprises administering to a subject in need thereof an effective amount of the compound N-{2-[(3-Bromophenyl)-4-fluorophenylamino]ethyl}acetamide (5p).

In an embodiment, the pain to be treated and/or treated is chronic and/or acute pain. The acute pain can for example be acute tonic pain, or pain relating to surgery, e.g. post-surgical pain, surgical pain, and/or trauma. In an embodiment, the pain is hyperalgesic pain or allodynic pain. In another embodiment, the pain is myalgic pain and/or inflammatory pain. In another embodiment the pain is neuropathic pain and/or nociceptive pain. In a further embodiment, the nociceptive pain is visceral pain or somatic pain, for example musculo-skeletal pain or post-traumatic pain. In another embodiment, the neuropathic pain is peripheral neuropathic pain or central neuropathic pain. In a further embodiment, the pain is back pain or joint pain. In yet another embodiment, the pain is head pain (e.g. headache). In another embodiment, the pain is pain associated with a disorder or condition. In an embodiment, the disorder or condition is chosen from fibromyalgia, irritable bowel syndrome, arthritis, ulcer, diabetic neuropathy, sciatica and migraine. In another embodiment, the ulcer is a gastric ulcer. In another embodiment the pain associated to vulvodynia. One or more types of pain can for example be treated and/or alleviated at the same time. One or more types of pain can for example be associated with a disorder or condition.

Yet a further aspect is a therapeutically effective composition for treating a condition mediated by at least one of MT₁ and MT₂ receptors, comprising one or more pharmaceutically acceptable excipients and a compound of formula:

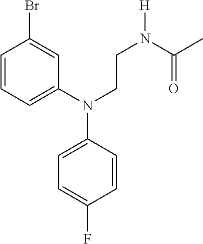 [SMILES: CC(=O)NCCN(c1ccc(F)cc1)c1cccc(Br)c1]

or a pharmaceutically acceptable salt thereof.

In an embodiment, the condition is associated with MLT activity. In another embodiment, the condition is chosen from sleep disorders, anxiety, depression, and chronobiological disorders. For example, the condition is a sleep disorder.

An aspect of the present disclosure is a pharmaceutical composition comprising a pharmaceutically acceptable excipient and a compound of formula:

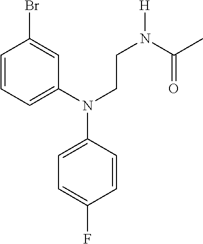 [SMILES: CC(=O)NCCN(c1ccc(F)cc1)c1cccc(Br)c1]

or a pharmaceutically acceptable salt thereof.

Another aspect is a method of interacting with at least one of MT₁ and MT₂ MLT receptor subtypes, comprising administering to a subject in need thereof an effective amount of a compound of Formula:

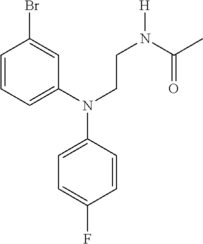 [SMILES: CC(=O)NCCN(c1ccc(F)cc1)c1cccc(Br)c1]

or a pharmaceutically acceptable salt thereof.

In an embodiment, the compound is a ligand to at least one of MLT receptor subtypes MT1 and MT2.

In another embodiment, the interacting treats conditions mediated by at least one of MT₁ and MT₂ receptors. In yet another embodiment, the condition is chosen from sleep disorders, anxiety, depression, and chronobiological disorders. For example, the condition is sleep disorders.

### Materials and Methods

Melting points were determined using a Buchi B-540 capillary melting point apparatus and are uncorrected. ¹H NMR spectra were recorded using a Bruker AVANCE 200 MHz spectrometer, using CDCl₃ as the reference solvent unless specified otherwise. Chemical shifts (δ scale) are reported in parts per million (ppm) relative to the central peak of the reference solvent. EI-MS spectra (70 eV) were taken using a Fisons Trio 1000 instrument. ESI-MS spectra were taken on a Waters Micromass ZQ instrument. Molecular ions (M⁺) and base peaks only are provided herein. Infrared spectra were obtained using a Nicolet Avatar 360 FT-IR spectrometer; absorbancies are reported in v (cm⁻¹). Elemental analyses for C, H and N were performed using a Carlo Erba analyzer. Column chromatography purifications were performed under “flash” conditions using Merck 230-400 mesh silica gel. Analytical thin-layer chromatography (TLC) was carried out on Merck silica gel 60 F₂₅₄ plates. All chemicals were purchased from commercial suppliers and used directly without any further purification.

In an embodiment, the compounds of Formula (I) may be prepared by procedures such as those illustrated in general Scheme 1 according to previously reported procedures. Other procedures, as well as variations thereof, could also be employed for preparing the compounds of Formula (I) and would be within the ability of one of ordinary skill in the art.

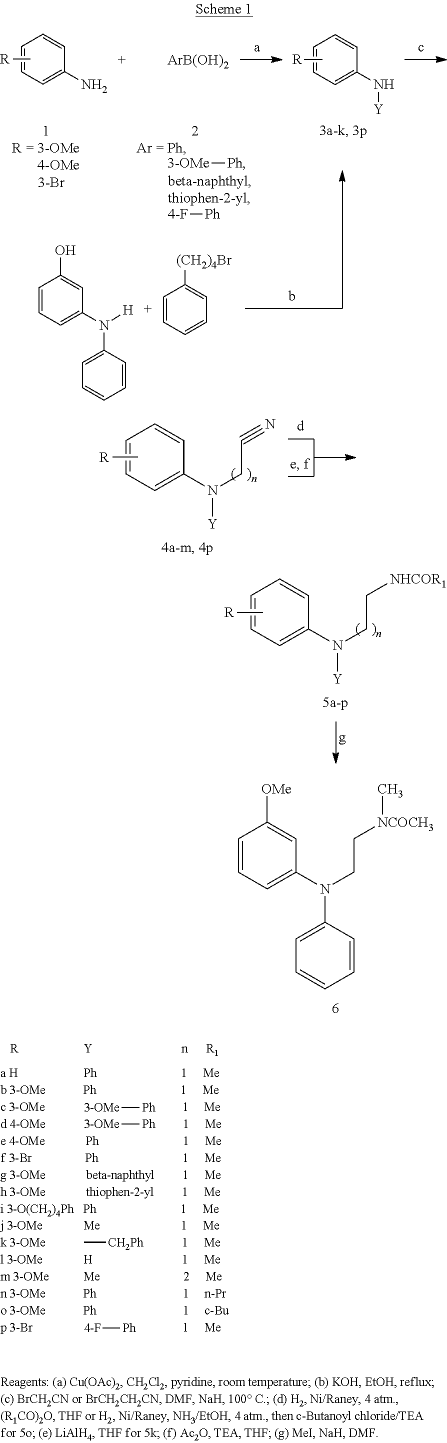

The (aminoalkyl)-amido derivatives (5a-p) were prepared by N-cyanoalkylation of the corresponding secondary amines (3a-k, 3p) with bromoacetonitrile or bromoproprionitrile in the presence of sodium hydride, followed by reduction of the intermediate nitriles (4a-m, 4p) and N-acylation of the crude N,N-disubstituted diamines with anhydrides, acid chloride or isocyanates (Scheme 1).

The starting anilines are commercially available, or were obtained by using previously reported procedures, such as the coupling reaction between an arylboronic acid (2) and an appropriate aniline (1) in the presence of cupric acetate and pyridine (Chan et al., 1998) by condensation of a suitable acetanilide with 3-bromoanisole (Akhavan-Tafti et al., 1988), or by palladium-catalyzed amination (Ji et al., 2003; Charles et al., 2005; Hartwig, 2008) of the suitable aniline with bromo-aryl compounds. Compound 6 was prepared by N-alkylation of 5b with Mel in the presence of NaH.

It is important to note that depending on the type of substituent on the phenyl ring (i.e. “R”), it is possible to further transform the compounds of Formula (I) into analogues thereof using procedures within the ability of one of ordinary skill in the art. For example, in order to prepare compounds of Formula (I) in which R is C₁-C₈ alkylthio, C₂-C₈ alkyloxy or phenylalkyloxy, the corresponding compound of Formula (I) in which R is OMe can be reacted with AlCl₃ or BBr₃ and the desired alkyl halide according to previously reported literature procedures (Caubere et al., 1994). Non-limiting examples of compounds prepared according to this procedure include N-{2-[(3-Butoxyphenyl)-methylamino]ethyl}acetamide: mp=68° C.; EI-MS 264 (M⁺), 192 (100); N-{2-[(3-Hexyloxyphenyl)-methylamino]ethyl}acetamide: mp=56° C.; EI-MS 292 (M⁺), 220 (100); and N-{2-{[3-(4-phenylbutoxy)phenyl)-methylamino]}ethyl}acetamide: mp=57° C.; EI-MS 340 (Re), 268 (100).

### Measurement of Melatonin Receptor Binding

The melatonin receptor binding affinities of the compounds of Formula (I) were determined using 2-[¹²⁵I]iodomelatonin as the labeled ligand in competition experiments on cloned human MT₁ and MT₂ receptors expressed in NIH3T3 rat fibroblast cells (Rivara et al., 2007; Rivara et al., 2009) or in CHO cells. To define the functional activity of the compounds of Formula (I) impedance assays in CHO cells expressing human-cloned MT₁ (Browning et al., 2000) or cAMP assays in CHO cells expressing human-cloned MT₂ receptors (Beresford et al., 1998) were performed.

Most compounds of the present disclosure (compounds of Formula (I)) have good to high affinity for MT₁ and/or MT₂ melatonin receptors, as determined in receptor binding assays, and show better affinity for the MT₂ than for the MT₁ receptor (PCT/CA2007/000055; Rivara et al., 2007; Rivara et al., 2009). For example, compound 5b exhibits better MT₂ affinity than melatonin, displays good MT₂-selectivity.

TABLE 1
Binding Affinity and Intrinsic Activity of some N-(substituted-anilinoalkyl)acylamines
(Formula I) for the Human MT₁ and MT₂ Melatonin Receptors Stably Expressed in CHO
Cells.
Formula I

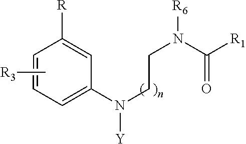

| | | | | | | | MT₁ | | MT₂ | |
|---|---|---|---|---|---|---|---|---|---|---|
| | R₃ | R | Y | n | R₁ | R₆ | pKᵢ^{a} | Activity^{b} | pKᵢ^{a} | Activity^{b} |
| MLT | — | — | — | — | — | — | 9.85 | A | 9.62 | A |
| 5b | OMe | H | Ph | 1 | Me | H | 8.38 | PA | 10.18 | PA |
| 5i | O(CH₂)₄Ph | H | Ph | 1 | Me | H | 7.45 | ANT | 6.48 | ANT |
| 5p | Br | H | 4-F—Ph | 1 | Me | H | 6.75 | ANT | 9.27 | PA |

^{a}pKi values were calculated from IC₅₀ values obtained from competition curves by the method of Cheng and Prusoff;
^{b}A = ago0nist; PA = partial agonist; ANT = antagonits.

Binding Affinity and Intrinsic Activity of other N-(substituted-anilinoalkyl)acylamines (Formula I) for the Human MT₁ and MT₂ Melatonin Receptors Stably Expressed in NIH3T3 Cells have been already reported (PCT/CA2007/000055; Rivara et al., 2007; Rivara et al., 2009).

### In Vivo Tests and Animals.

Animals: Wistar rats (150-250 g, Charles-River Saint-Constant, Quebec, Canada) were used for the formalin test, hot plate test and L5-L6 spinal nerves ligature model. The animals were housed at constant room temperature and humidity under a 12 h light/dark cycle (lights on at 7 AM). Food and water were available ad libitum.

Hot plate test: The hot-plate test was performed by using an electronically controlled hot-plate (Ugo Basile, Italy). The initial temperature was 38° C. A near linear increase in temperature (3° C. per min) was obtained by turning the heating adjustment to maximum. The temperature at which the fast hindpaw lick occurred was recorded as the nociceptive end-point. If no hindpaw lick was observed, the test was terminated at 52° C., and this cut-off value was used in the analysis. The plate was wiped with a wet cloth, and a fan located behind the plate allowed rapid cooling so that testing could be conducted every 7.5 min.

Formalin Test: Antinociception was assessed using the formalin test (Dubuisson and Dennis, 1977). Rats were placed in open Plexiglas observation chambers for 60 min to allow them to acclimatize to their surroundings; then they were removed for formalin administration. Rats were gently restrained while the dorsal surface of the right hindpaw was injected with 50 ml of 1% formalin (37% formaldehyde solution further diluted in saline solution) with a 30-gauge needle. The animals were returned to the chambers and nociceptive behavior was observed immediately after formalin injection. Mirrors were placed in each chamber to enable unhindered observation. Licking/biting behavior directed to the formalin injected paw was scored in 5 min intervals considering number and duration of the licking. Cumulative behavior for the total duration of the test as well as the first (0-10 min) and second phase (20-50 min) was then analyzed (Sufka et al., 1998).The initial acute phase (0-10 min) was followed by a relatively short quiescent period, which was then followed by a prolonged tonic response (15-60 min). The first phase is considered to be the result of direct stimulation of nociceptors, whereas inflammation process contributes to phase 2 activity (Hunskaar and Hole, 1987). At the end of the experiment the rats were sacrificed in a CO2 chamber.

L5-L6 Spinal Nerves Ligature: Nerve injury results in abnormal pain perception, known as neuropathic pain, which is associated with hyperalgesia (high pain sensibility) and allodynia (pain perception induced by innocuous stimuli) (Campbell and Meyer, 2006). The rat model for neuropathic pain develop by Kim and Chung (Kim and Chung, 1992) was used in which the L5 and L6 spinal nerves were ligated and two weeks later allodynia was measured by paw stimulation with von Frey filaments. Briefly, animals were anesthetized with a mixture of ketamine/xylazine/acepromazine (i.p.). Following surgical preparation and exposure of the dorsal vertebral column, the left L5 and L6 spinal nerves were exposed and tightly ligated with 3.0 chromic catgut suture distal to the dorsal root ganglion. For sham-operated rats, the nerves were exposed but not ligated. The incisions were closed using 3.0 vicryl sutures, and the animals were allowed to recover for 13 days. Rats exhibiting motor deficiency (such as paw dragging) were excluded from testing (less than 5%).

On day 14 after surgery, rats were singularly placed in a test chamber (clear plastic, wire mesh-bottomed cage) and allowed to acclimatize for 30-40 min. Von Frey filaments (Stoelting, Wood Dale, Ill., USA) were used to measure the 50% paw withdrawal threshold using the up and down method by (Chaplan et al., 1994). A series of filaments, starting with one that had a buckling weight of 2 g, were applied in consecutive sequence surface and on the left hind paw with a pressure causing the filament to buckle. Lifting of the paw indicated a positive response and prompted the use of the next weaker filament, whereas absence of paw withdrawal after 5 s indicated a negative response and prompted the next filament of increasing weight. This paradigm continued for four more measurements after the initial change of the behavioral response, or until five consecutive negative (assigned a score of 15 g), or four consecutive positive (assigned a score of 0.25 g) responses. The resulting score were used to calculate the 50% response threshold by using the formula proposed by Dixon et al., 1980.

Allodynia was considered to be present when paw withdrawal thresholds were less than 4 g. (Chaplan et al., 1994). All nerve-ligated rats were verified to be allodynic, responding to a stimulus of less than 4 g. Rats without allodynia were excluded. Following the determination of the basal response, tactile allodynia was assessed at 0.5, 1, 2, 3, 4, 5, 6, 7 and 8 h post-administration for each treatment below described.

Rats were assigned to receive increasing doses of compound 5b (10, 20, 40 mg/kg) or compound 5p (10, 20, 40 mg/kg). The positive control used was gabapentin (GBP, 100 mg/kg) and melatonin (MLT, 150 mg/kg) treatment was used also for comparison. Another group of rats received only the vehicle (VEH) consisting on a 70% DMSO/saline solution (0.9% NaCl). All treatments were administered in a single subcutaneous injection with a volume of 1 ml. Rats were tested at 0 h (basal withdrawal threshold) and 0.5, 1, 2, 3, 4, 5, 6, 7 and 8 h post-treatment administration.

Spared nerve injury (SNI) of the sciatic nerve and measurement of allodynia with the dynamic plantar aesthesiometer. SNI was performed according to the method of Decosterd and Woolf (2000). Rats were anaesthetized with sodium pentobarbital (50 mg/kg i.p.). The sciatic nerve was exposed at mid-thigh level distal to the trifurcation and freed of connective tissue; the three peripheral branches (sural, common peroneal, and tibial nerves) of the sciatic nerve were exposed without stretching nerve structures. Both tibial and common peroneal nerves were ligated and transected together. The sham procedure consisted of the same surgery without ligation and transection of the nerves. Fourteen days after SNI, mechanical allodynia was measured using the dynamic plantar aesthesiometer (Ugo Basile, Varese, Italy). Each rat was placed and allowed to move freely in one of the two compartments of the enclosure positioned on a metal grid surface. A mechanical stimulus was delivered to the plantar surface of the rat's hind paw through the metal grid by a steel filament (Von Frey-type), connected to a movable touch-stimulator unit exerting an increasing force of 3 g per second. The force inducing paw withdrawal was recorded to the nearest 0.1 g. Nociceptive responses for mechanical sensitivity (mechanical withdrawal threshold) were measured in grams before and after vehicle or drug administration by an experimenter blind to the treatments. A single trial at each time point was performed on the ipsilateral hind paw to SNI surgery for each rat. Nociceptive responses for mechanical sensitivity were expressed as mean±SEM (g). Groups of 6-7 rats per treatment were used, with each animal being used for one treatment only. As previously described for the L5-L6 model, SNI rats were randomly assigned to receive a single s.c. injection of 5b or 5p at the doses of 10, 20 or 40 mg/kg and the effects of both drugs were compared with those produced by MLT (150 mg/kg), GBP (100 mg/kg) and VEH administration.

Statistical Analysis: Data analysis was done using SigmaPlot 12 (Systat Software, Inc.). Two-way ANOVA for repeated measures was used to analyze data from L5-L6 models using treatments (between) and testing time (within) as factors. One-way ANOVA was used to analyze AUC in the L5-L6 spinal nerves ligation model. One-way ANOVA was used for formalin and hot-plate tests. Post-hoc analyses were performed using Bonferroni t-test comparisons or Student-Newman-Keuls (SNK) test. All data are expressed as mean±Standard Error of the Mean (SEM). P<0.05 was considered significant.

### Results

In Vivo Tests in Animals. The hot plate, formalin test, the L5-L6 sciatic nerves ligature and the SNI in rats were employed to evaluate the anti-pain properties of compounds of Formula (I). The following results were obtained using compounds 5b and 5p.

Effects of 5b and 5p on the hot plate test: The effect of the different doses of 5b (10-40 mg/kg) at 30 min, 1 and 4 hours post treatments is shown in FIG. 1A. Two-way ANOVA revealed a significant main effect for treatment (P<0.001). Post-hoc analysis at 30 min revealed that the dose of 20 mg/kg of 5b induced a significant increase of withdrawal temperature compared with vehicle (p<0.01) and with 5b at the dose of 10 mg/kg (p<0.001) (FIG. 1A). The analysis at 1 hour revealed an increase of withdrawal temperature for the doses of 20 mg/kg (p<0.001) and 40 mg/kg (p<0.01) of 5b versus control. At the same time, the doses of 5b of 20 and 40 mg/kg increased withdrawal temperature in comparison with 5b 10 mg/kg treated rats (p<0.001 and p<0.01, respectively). Post-hoc analysis at 4 hours revealed that 5b at doses of 20 and 40 mg/kg induced a significant increase of withdrawal temperature compared with vehicle (p<0.001) and with the dose of 5b 10 mg/kg (p<0.001), respectively (FIG. 1A).

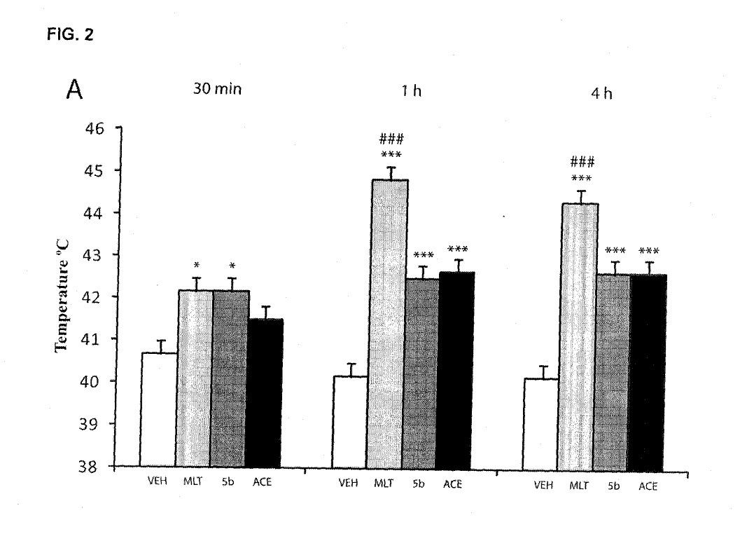

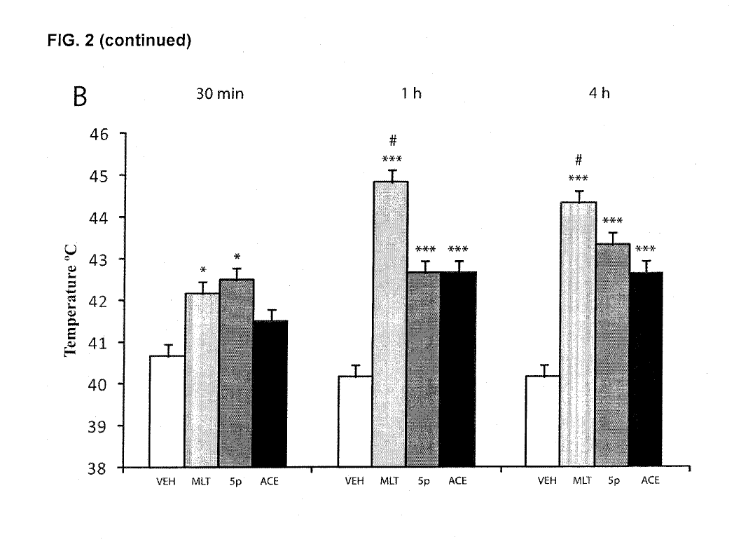

The effects of MLT (150 mg/kg), 5b (20 mg/kg) and ACE (200 mg/kg) on the hot plate test are reported in FIG. 2A. Two-way ANOVA followed by Post hoc comparisons showed significant increase of the withdrawal temperature of 5b 20 mg/kg (p<0.05) and MLT (p<0.05) treated rats versus vehicle after 30 min post treatment. The analysis after 1 hour revealed an increase of withdrawal temperature for the 5b at 20 mg/kg (p<0.001), MLT (p<0.001) and acetaminophen (p<0.001) versus control treated rats. At the same time, MLT increased the withdrawal temperature in comparison with 5b 20 mg/kg (p<0.001) and acetaminophen (p<0.001). Post-hoc analysis after 4 hours revealed that 5b 20 mg/kg, MLT and acetaminophen increased the withdrawal temperature compared with vehicle (p<0.001). MLT treated rats also increased the withdrawal temperature compared with 5b, 20 mg/kg (p<0.001) and acetaminophen (p<0.001), respectively (FIG. 2A).

The effect of the different doses of 5p (10-40 mg/kg) at 0.5, 1 and 4 hours post treatments is shown in FIG. 1B. Two-way ANOVA revealed a significant main effect for treatment (P<0.001). Post-hoc analysis after 30 min revealed that the dose of 20 mg/kg of 5p induced a significant increase of withdrawal temperature compared with vehicle (p<0.001), 5p 10 mg/kg (p<0.001) and with 5p 40 mg/kg (p<0.001) treated rats (FIG. 1B). The analysis after 1 hour revealed an increase of withdrawal temperature for the doses of 20 and 40 mg/kg of 5p versus vehicle (p<0.001) and 5p 10 mg/kg treated rats (p<0.001). Post-hoc analysis at 4 hours revealed that 5p at doses of 20 and 40 mg/kg induced a significant increase of withdrawal temperature compared with vehicle (p<0.001) and 5p 10 mg/kg (p<0.001), respectively (FIG. 1B).

The effects of MLT (150 mg/kg), 5p (20 mg/kg) and acetaminophen (200 mg/kg) on the hot plate test are reported in FIG. 2B. Post hoc comparisons showed a significant increase of the withdrawal temperature of 5p 20 mg/kg (p<0.05) and MLT (p<0.05) treated rats versus vehicle after 30 min post treatment. The analysis at 1 hour revealed an increase of withdrawal temperature for the 5p at 20 mg/kg (p<0.001), MLT (p<0.001) and acetaminophen (ACE) (p<0.001) versus control treated rats. At the same time, MLT increased the withdrawal temperature in comparison with 5p 20 mg/kg (p<0.01) and acetaminophen (p<0.01). Post-hoc analysis after 4 hours revealed that 5p 20 mg/kg, MLT and acetaminophen increase the withdrawal temperature compared with vehicle (p<0.001). MLT treated rats also increase the withdrawal temperature compared with 5p 20 mg/kg (p<0.05) and acetaminophen (p<0.01), respectively (FIG. 2B).

Formalin Test: Subcutaneous injection of 1% formalin into the hindpaw produced a biphasic licking behavior. The first phase started immediately after formalin injection and declined gradually in about 10 min. The second phase initiated 15 min after formalin injection and it reached a maximum effect between 30 and 40 min declining gradually at about 60 min. The effect of the different doses of 5b (10-40 mg/kg) during the first phase, second phase and total time is shown in FIG. 3A. One-way ANOVA of the different doses of 5b (10-40 mg/kg) revealed significant differences between treatments in cumulative licking time in the animals during the first phase (p<0.01), second phase (p<0.01,) and the total (p<0.001) time. Post hoc analysis for the first phase revealed a decrease in the licking time with 5b at 10 (p<0.05), 20 (p<0.05), 40 (p<0.01) mg/kg compared with vehicle (VEH, 70% DMSO in saline solution) (FIG. 3A). Post hoc analysis for the second phase exposed that 5b at 20 (p<0.05) and 40 mg/kg (p<0.001) decreased the licking time compared with vehicle. 5b at 40 mg/kg decreased the licking time versus 5b at 20 (p<0.05) and 10 (p<0.05) mg/kg doses. Post hoc analysis of the total time showed a reduction of the licking time with the pre-treatment with 5b at 10 (p<0.05), 20 (p<0.01) and 40 (p<0.001) compared with vehicle group. 5b at 40 mg/kg decreased the licking time versus 5b at 20 (p<0.05) and 10 (p<0.05) mg/kg (FIG. 3A).

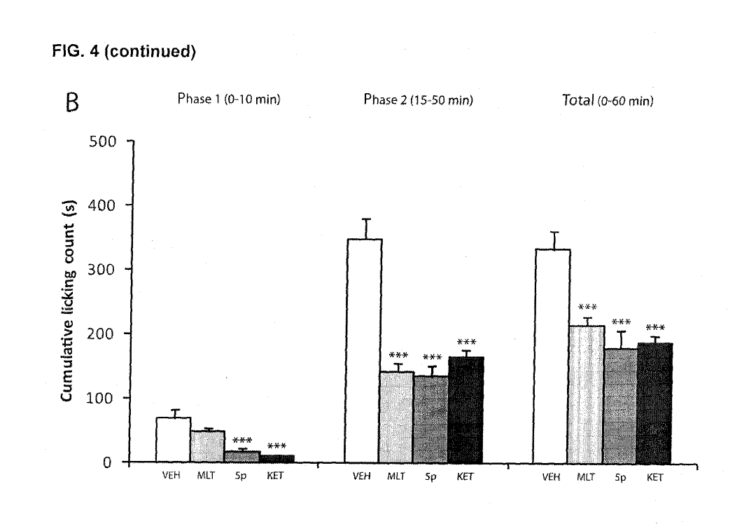

The effects of MLT (150 mg/kg), 5b (20 mg/kg) and ketoprofen (3 mg/kg) in the formalin test are reported in FIG. 4A. One-way ANOVA revealed a significant difference in cumulative licking time between treatments in the animals during the first (p<0.001), second (p<0.001,) and the total (F_{3,23}=10.86, p<0.001) time. Post hoc analysis for the first phase revealed a decrease in the licking time with 5b 20 mg/kg (p<0.01), Melatonin (p<0.001) and Ketoprofen (p<0.001) compared with vehicle treated rats (FIG. 4A). The analysis of the second phase showed a decrease of the licking time with 5b 20 mg/kg (p<0.001), MLT (p<0.001) and Ketoprofen (p<0.001) treated rats compared with vehicle. Post hoc analysis for the total time revealed a decrease in the licking time with 5b 20 mg/kg (p<0.001), MLT (p<0.001) and Ketoprofen (p<0.001) treated rats compared with vehicle (FIG. 4A).

One-way ANOVA of the different doses of 5p (10-40 mg/kg) revealed a significant difference between treatments in cumulative licking time in the animals during the first (p<0.01), second (p<0.001,) and the total (p<0.05) time (FIG. 3B). Post hoc analysis for the first phase revealed a decrease in the licking time with 5p at 10 (p<0.05), 20 (p<0.05), 40 (p<0.01) mg/kg compared with vehicle treated rats (FIG. 3B). Post hoc analysis for the second phase revealed that 5p at 10 (p<0.01), 20 (p<0.001) and 40 (p<0.01) mg/kg decreased the licking time compared with vehicle. Post hoc analysis of the total time showed a reduction of the licking time with the pre-treatment with 5p at 20 (p<0.01) and 40 (p<0.05) mg/kg compared with vehicle group.

The effects of MLT (150 mg/kg), 5p (20 mg/kg) and ketoprofen (3 mg/kg) in the formalin test are reported in FIG. 4B. One-way ANOVA revealed a significant difference in cumulative licking time between treatments in the animals during the first phase (p<0.001), second phase (p<0.001,) and the total time (p<0.001). Post hoc analysis for the first phase revealed a decrease in the licking time with 5p 20 mg/kg (p<0.001) and ketoprofen (p<0.001) compared with vehicle treated rats (FIG. 4B). The analysis of the second phase showed a decrease of the licking time with 5p 20 mg/kg (p<0.001), MLT (p<0.001) and ketoprofen (p<0.001) treated rats compared with vehicle. Post hoc analysis for the total time revealed a decrease in the licking time with 5p 20 mg/kg (p<0.001), MLT (p<0.001) and ketoprofen (p<0.001) treated rats compared with vehicle (FIG. 4B).

L5-L6 Spinal Nerves Ligature: The sensitivity to von Frey filament stimulation at the plantar surface of the hindpaw of both nerve ligated and sham animals was determined to in order to test whether spinal nerve ligation led to the development of neuropathic pain. The data indicate that the nerve ligation induces a reduction in the hindpaw withdrawal threshold to the mechanical stimulation to a level considered allodynic (4 g) (Chaplan, 1994). Systemic administration of 5b reversed tactile allodynia induced by spinal nerve ligation, increasing the paw withdrawal threshold in a dose-dependent manner. A reduction of the mechanical allodynia above the threshold was induced by the administration of 5b (20 mg/kg and 40 mg/kg) but not by 5b at 10 mg/kg and 5 mg/kg (p>0.05 vs 20 and 40 mg/kg. FIG. 5A). The maximal antiallodynic effect was reached with 5b (20 mg/kg) and greater doses did not produce a greater antiallodynic effect.

The time course effect of the different doses of 5b (5-40 mg/kg) is shown in FIG. 5A. Two-way ANOVA analysis revealed a significant interaction between treatment and time of testing (p<0.001). Post-hoc analysis revealed that 5b at 20 (p<0.001) and 40 mg/kg (p<0.001) induced a significant increase of withdrawal threshold starting at 1 and 0.5 h post-administration, respectively (FIG. 5A). The highest increase in withdrawal threshold was observed at 3 h post-administration for the dose of 20 mg/kg (419% from basal) and at 2 h post-administration for the dose of 40 mg/kg (395% from basal), and lasted for up to 5 h and 6 h post-administration, respectively. A significant interaction among treatments and time of test was also detected when the effects of 5b (20 mg/kg) were compared with MLT and GBP treated rats and with rats receiving the MT₂ antagonist 4P-PDOT (10 mg/kg) prior to 5b (20 mg/kg) (p<0.001; FIG. 5B). Post-hoc analysis of the area under the curve (AUC, FIG. 5C) also showed that 5b (20 and 40 mg/kg) was superior to MLT (150 mg/kg) (p<0.001). In particular, AUC's post-hoc analyses across 8 h indicated that, compared to VEH-treated rats (measured as mean±SEM, weight (g) of mechanical allodynia, 36.24±1.8 g), 5b treatment induced a significant dose-dependent anti-allodynia effect (5b, 5 mg/kg: 81.11±4.8 g; 10 mg/kg: 70.24±4.23 g; 20 mg/kg: 219.12±24.13 g; 40 mg/kg: 220.13±13.65 g; p<0.001, FIG. 5C). The anti-allodynia effect of 5b at the doses of 20 and 40 mg/kg was comparable to the AUC produced by administration of a 100 mg/kg dose of GBP (249.33±22.1 g) and were greater than the effects produced by MLT administration (150 mg/kg=131.75±7.9, p<0.001) (FIG. 5C).

Similar results were obtained with the administration of 5p, showing a reduction of the mechanical allodynia at 20 mg/kg and 40 mg/kg. The time course of the effects of 5p is shown in FIG. 6A. Two-way ANOVA analysis revealed a significant interaction between treatment and time of testing (p<0.001). Compared with basal withdrawal threshold (0 h), 5p at the doses of 10 mg/kg (p<0.001), 20 mg/kg (p<0.001) and 40 mg/kg (p<0.001) increased paw withdrawal threshold starting at 1 h post-administration. The effect of 10 mg/kg dose lasted up to 3h, but the effects of the doses of 20 and 40 mg/kg remained stable for up to 6 h post-administration with a maximum increase of threshold at 5 h (20 mg/kg: 394% from basal; 40 mg/kg: 427.65% from basal). Compared with VEH-treated rats, 5p at 10 mg/kg dose increased paw withdrawal threshold at 1, 2, 3, 4 h post-administration, whereas the dose of 20 and 40 mg/kg increased it from 1 h to 6 h post-administration (see FIG. 6A).

Effects of 5p are similar to those observed in rats treated with GBP at 100 mg/kg (FIG. 6B and FIG. 6C). GBP treatment increased paw withdrawal threshold from 1 h to 6 h compared with basal withdrawal threshold (0 h) and from 1 h to 7 h compared with VEH treated rats. The maximum withdrawal threshold increase induced by GBP was observed at 4 h (420% from basal). MLT treated rats showed an increased withdrawal threshold from 1 h to 3 h compared with their baseline threshold and with VEH group, with a maximum increase in threshold at 2 h post-treatment (335% from baseline). Pre-treatment with 4P-PDOT blocked the effects of 5p during the 8 h of testing (FIG. 6B).

Analysis of the AUC during the 8 h of testing indicated that, compared to VEH-treated rats (measured as mean±SEM, weight (g) of mechanical allodynia 36.24±1.8 g), 5p treatments induced a significant dose-dependent anti-allodynia effect (5p, 5 mg/kg: 89.03±6.33 g; 10 mg/kg: 127.75±12.08 g; 20 mg/kg: 245.9±19.9 g; 40 mg/kg: 230.33±17.69 g; p<0.001, FIG. 6C). All the doses from 5 to 40 mg/kg were significant different compared to vehicle (FIG. 6C). The effect of 5p at the doses of 20 and 40 mg/kg were comparable with the AUC produced by administration of a 100 mg/kg dose of GBP (249.33±22.1 g) and had a greater antiallodynic effect than that produced by MLT administration (150 mg/kg=131.75±7.9, p<0.001).

SNI model: SNI of the sciatic nerve resulted in a significant decrease in mechanical withdrawal threshold in the ipsilateral side of SNI rats, though not on the contralateral side, 7 days after surgery. Administration of VEH did not change withdrawal threshold in the SNI rats (FIG. 7A,B). The analysis of the dose-response curve of the effects of 5b or 5p on the withdrawal threshold in the SNI rats (FIG. 7A and FIG. 7B, respectively) showed a significant interaction between treatment and testing time for both 5b (p<0.01) and 5p (p<0.001) and an effect of 5b and 5p for treatment (p<0.001). A significant reduction of the mechanical allodynia was caused by the treatment with 5b at the doses of 20 (p<0.05) and 40 mg/kg (p<0.001) but not at the dose of 10 mg/kg (FIG. 7A); and with 5p at the dose of 20 mg/kg and 40 mg/kg (p<0.001), 5p (20 and 40 mg/Kg) was effective up to 7 h after administration and reversed almost completely mechanical allodynia (FIG. 7B). In addition, there are no differences in the effects elicited by the dose of 20 and 40 mg/kg. The effects of 5b and 5p (20 mg/kg) with those of MLT (150 mg/kg) and GBP (100 mg/kg) on mechanical allodynia in SNI rats were then compared (FIG. 7C). The effects produced by 5p were greater than those produced by GBP (p<0.01), MLT (p<0.001), and VEH (p<0.001). The decrease in mechanical allodynia yielded by MLT was apparent up to 5 h after administration, whereas the effect yielded by GBP, similarly to 5p, was apparent up to 7 h after administration (FIG. 7C). The effect of MLT was of a lower magnitude than that produced by GBP (p<0.001).

It is to be understood that the disclosure is not limited in its application to the details of construction and parts illustrated in the accompanying drawings and described hereinabove. The disclosure is capable of other embodiments and of being practiced in various ways. It is also understood that the phraseology or terminology used herein is for the purpose of description and not limitation. Hence, although the present disclosure has been described hereinabove by way of preferred embodiments thereof, it can be modified, without departing from the spirit, scope and nature of the subject disclosure as defined in the appended claims.

### BIBLIOGRAPHY

Akhavan-Tafti et al. Tetrahedron Letters 1988, 63, 930
Ambriz-Tututi M. et al. Life Sci. 2009, 84, 489-498
Ambriz-Tututi M, Granados-Soto V. Pain, 2007, 132, 273-280.
Bruel-Jungerman E, Rampon C, Laroche S. Rev Neurosci 2007; 18:93-114.
Brunner P, Sozer-Topcular N, Jockers R, et al. Eur J Histochem 2006; 50:311-16.
Campbell J., Meyer R. Neuron 2006, 52, 77-92.
Caubere C., Cauber P., Renard P. et al. Tetrahedron 1994, 50, 13433-13448.
Chan D. M. T. et al. Tetrahedron Lett 1998, 39, 2933.
Chaplan, S, et al J Neurosci Methods 1994, 53, 55-63.
Charles M. D., Schultz P., Buchwald S. L. Org. Lett. 2005, 7, 3965-3968.
Decosterd I. & Woolf C. J., Pain, 2000, 87, 149-158
Deng W, Aimone J B, Gage F H. Nat Rev Neurosci 2010; 11:339-50.
Dietrich K., Birkmeier S., Schleinitz D., et al. Diabet Med 2011, 28, 1373-1380.
Dixon W. J. Annu. Rev. Pharmacol. Toxicol. 1980, 20, 441-462
Dubocovich M. L. et al. Pharmacol Rev. 2010, 62, 343-380
El-Sherif Y, Tesoriero J, Hogan M V, et al. J Neurosci Res 2003, 72:454-60.
Ekmekcioglu C., Thalhammer T., Humpeler S., et al. J Pineal Res 2003, 35, 40-44.
Ha E., Choe B. K., Jung K. H., et al. J Pineal Res 2005, 39, 201-205.
Hartwig J. F. Acc. Chem. Res. 2008, 41, 1534-1544.
Hunskaar S. and Hole K. Pain 1987, 30, 103-114.
Hussein S. A. et al. J Pineal Res. 2011, 50, 267-271.
Jarrat J. Anaesth Intensive Care 2011, 39, 171-181.
Ji J., Li T., Bunnelle W. H. Org. Lett. 2003, 5, 4611-4614
Kim S. and Chung J. Pain 1992, 50, 355-363.
Larson J, Jessen R E, Uz T, et al. Neurosci Lett 2006; 393:23-26.
Nosjean O. et al. J Biol Chem 2000, 275, 31311-31317
Pang C. S. et al. Life Sci 2001, 68, 943-951.
Paz Romero, M. et al FASEB J. 1998, 12, 1401-1408
PCT/CA2007/000055.
Prokopenko I., Langenberg C., Florez J. C., et al. Nat Genet 2009, 41, 77-81.
Rivara S. et al. J Med Chem. 2007, 50, 6618-6626.
Rivara S. et al. ChemMedChem. 2009, 4, 1746-1755.
Savaskan E, Ayoub M A, Ravid R, et al. J Pineal Res 2005; 38:10-16.
Savaskan E, Jockers R, Ayoub M, et al. Curr Alzheimer Res 2007; 4:47-51.
Simonneaux V., Ribelayga C. Pharmacol Rev 2003, 55, 325-395.
Song J. Y., Wang H. J., Ma J., et al. Obes Facts 2011, 4, 197-203.
Stone L. S., Molliver D. C. Molecular Interventions 2009, 9, 234-251.
Sufka K. J. et al. Eur J. Pain 1998, 2, 351-358.
Squire L R. Psychol Rev 1992; 99:195-231.
Wan Q. et al. Neurosci Lett 1996, 204, 77-80.
Wan Q, Man H Y, Liu F, et al. Nat Neurosci 1999, 2, 401-403.
Wang L M, Suthana N A, Chaudhury D, et al. Eur J Neurosci 2005; 22:2231-7.
Wilhelmsen M. et al. J Pineal Res 2011, 51, 270-277.
Zahn P. K. et al. J Pineal Research 2003, 35, 24-31.

1.-71. (canceled)

72. A method of treating pain comprising administering to a subject in need thereof a compound of Formula I: 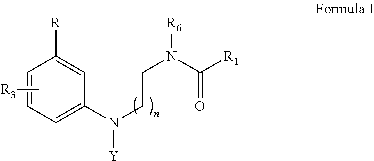
or a pharmaceutically acceptable salt thereof, wherein:
n is 1 or 2;
m is 0, 1 or 2;
p is 0, 1, 2, 3, 4, 5, 6, 7 or 8;
v is 2 or 3;
A is aryl or heteroaryl;
Z is O, S or NR₈;
Y is chosen from hydrogen, aryl, heteroaryl, C₁-C₆ alkyl, C₃-C₆ cycloalkyl, and 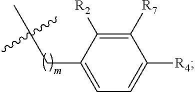
R is chosen from hydrogen, hydroxyl, —OCF₃, CF₃, C₁-C₈ alkyl, C₁-C₈ alkyloxy, C₁-C₈ alkylthio, halogen and —Z—(CH₂)ₚ-A;
R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl;
R₂ is chosen from hydrogen, C₁-C₄ alkyl, C₁-C₄ alkyloxy, OCF₃, CF₃, hydroxyl, and halogen;
R₃ is chosen from hydrogen, C₁-C₄ alkyl, C₁-C₄ alkyloxy, OCF₃, CF₃, hydroxyl and halogen;
R and R₃ may be connected together to form an —O—(CH₂)ᵥ bridge representing with the carbon atoms to which they are attached a 5- or 6-membered heterocyclic ring system;
R₄ is chosen from hydrogen, C₁-C₄ alkyl, C₁-C₄ alkyloxy, OCF₃, CF₃, hydroxyl, and halogen;
R₆ is chosen from hydrogen and C₁-C₆ alkyl;
R₇ is chosen from hydrogen, C₁-C₄ alkyl, C₁-C₄ alkyloxy, OCF₃, CF₃, hydroxyl and halogen;
R₈ is chosen from hydrogen and C₁-C₄ alkyl.

73. The method of claim 72, wherein:
n is 1 or 2;
m is 0, or 1;
p is 0, 1, 2, 3, or 4;
A is phenyl;
Z is O;
Y is chosen from hydrogen, methyl, [symbol] -naphthyl, thiophene-3-yl, and 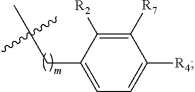
R is chosen from hydrogen, methoxy, Br and —Z—(CH₂)ₚ-A;
R₁ is chosen from methyl, propyl and cyclobutyl;
R₂ is hydrogen;
R₃ is chosen from hydrogen, halogen and methoxy;
R₄ is hydrogen or halogen;
R₆ is hydrogen or methyl;
R₇ is hydrogen, hydroxy or methoxy.

74. The method of claim 72, wherein the compound is chosen from N-[2-(diphenylamino)ethyl]acetamide (5a), N-{2-[(3-Methoxyphenyl)-phenylamino]ethyl}acetamide (5b), N-[2-(bis-3-methoxyphenylamino)ethyl]acetamide (5c), N-{2-[(4-Methoxyphenyl)-3-methoxyphenylamino]ethyl}acetamide (5d), N-{2-[(4-Methoxyphenyl)-phenylamino]ethyl}acetamide (5e), N-{2-[(3-bromophenyl)-phenylamino]ethyl}acetamide (5f), N-{2-[(3-Methoxyphenyl)-β-naphthylamino]ethyl}acetamide (5g), N-{2-[(3-methoxyphenyl)(thiophen-2-yl)amino]ethyl}acetamide (5h), N-{2-[(3-pheny/butoxyphenyl)-phenyl-amino]ethyl}acetamide (5i), N-{2-[(3-Methoxyphenyl)-methylamino]ethyl}acetamide (5j), N-{2-[(3-Methoxyphenyl)-benzylamino]ethyl}acetamide (5k), N-{2-[(3-Methoxyphenyl)-amino]ethyl}acetamide (5l), N-{3-[(3-Methoxyphenyl)-methylamino]propyl}acetamide (5m), N-{2-[(3-Methoxyphenyl)-phenylamino]ethyl}butanamide (5n), N-{2-[(3-Methoxyphenyl)-phenylamino]ethyl}cyclobutancarboxamide (5o), N-{2-[(3-Bromophenyl)-4-fluorophenylamino]ethyl}acetamide (5p), N-Methyl-N-{2-[(3-methoxyphenyl)-phenylamino]ethyl}acetamide (6), N-{2-[(3-butoxyphenyl)-methylamino]ethyl}acetamide, N-{2-[(3-hexyloxyphenyl)-methylamino]ethyl}acetamide, and N-{2-{[3-(4-phenylbutoxy)phenyl)-methylamino]}ethyl}acetamide.

75. The method of claim 72, wherein the compound is N-{2-[(3-Methoxyphenyl)-phenylamino]ethyl}acetamide (5b).

76. The method of claim 72, wherein the compound is N-{2-[(3-Bromophenyl)-4-fluorophenylamino]ethyl}acetamide (5p).

77. The method of claim 72, wherein the pain is chosen from chronic pain and acute pain.

78. The method of claim 72, wherein the pain is chosen from myalgic pain, inflammatory pain, neuropathic pain and/or nociceptive pain.

79. The method of claim 77, wherein the acute pain is post-surgical pain, acute tonic pain, and/or trauma pain.

80. The method of claim 72, wherein the pain is back pain, joint pain and/or head pain.

81. The method of claim 78, wherein the nociceptive pain is visceral pain or somatic pain and/or the neuropathic pain is peripheral neuropathic pain or central neuropathic pain.

82. The method of claim 72, wherein the pain is associated with a disorder or condition and the disorder or condition is chosen from fibromyalgia, irritable bowel syndrome, arthritis, ulcer, diabetic neuropathy, sciatica and migraine.

83. The method of claim 72, wherein the treatment induces an analgesic effect.

84. The method of claim 72, wherein the treatment induces an antinociceptive effect.

85. The method of claim 72, wherein the treatment induces an antiallodynic effect.

86. The method of claim 72, wherein the treatment induces an anesthetic effect.

87. The method of claim 72, wherein the treatment induces an antihyperalgesic effect.

88. A method of treating pain comprising administering to a subject in need thereof a therapeutically effective composition comprising one or more pharmaceutically acceptable excipients and a compound of Formula I or a pharmaceutically acceptable salt thereof: 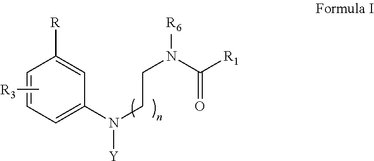
wherein:
n is 1 or 2;
m is 0, 1 or 2;
p is 0, 1, 2, 3, 4, 5, 6, 7 or 8;
v is 2 or 3;
A is aryl or heteroaryl;
Z is O, S or NR₈;
Y is chosen from hydrogen, aryl, heteroaryl, C₁-C₆ alkyl, C₃-C₆ cycloalkyl, and 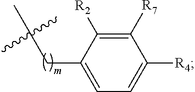
R is chosen from hydrogen, hydroxyl, —OCF₃, CF₃, C₁-C₈ alkyl, C₁-C₈ alkyloxy, C₁-C₈ alkylthio, halogen and —Z—(CH₂)ₚ-A;
R₁ is chosen from C₁-C₄ alkyl, C₃-C₆ cycloalkyl, CF₃, hydroxy-substituted C₁-C₄ alkyl, hydroxy-substituted C₃-C₆ cycloalkyl, and NHR₅, wherein R₅ is H, C₁-C₃ alkyl or C₃-C₆ cycloalkyl;
R₂ is chosen from hydrogen, C₁-C₄ alkyl, C₁-C₄ alkyloxy, OCF₃, CF₃, hydroxyl, and halogen;
R₃ is chosen from hydrogen, C₁-C₄ alkyl, C₁-C₄ alkyloxy, OCF₃, CF₃, hydroxyl and halogen;
R and R₃ may be connected together to form an —O—(CH₂)ᵥ bridge representing with the carbon atoms to which they are attached a 5- or 6-membered heterocyclic ring system;
R₄ is chosen from hydrogen, C₁-C₄ alkyl, C₁-C₄ alkyloxy, OCF₃, CF₃, hydroxyl, and halogen;
R₆ is chosen from hydrogen and C₁-C₆ alkyl;
R₇ is chosen from hydrogen, C₁-C₄ alkyl, C₁-C₄ alkyloxy, OCF₃, CF₃, hydroxyl and halogen;
R₈ is chosen from hydrogen and C₁-C₄ alkyl.

89. The method of claim 88, wherein:
n is 1 or 2;
m is 0, or 1;
p is 0, 1, 2, 3, or 4;
A is phenyl;
Z is O;
Y is chosen from hydrogen, methyl, [symbol] -naphthyl, thiophene-3-yl, and 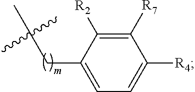
R is chosen from hydrogen, methoxy, Br and —Z—(CH₂)ₚ-A;
R₁ is chosen from methyl, propyl and cyclobutyl;
R₂ is hydrogen;
R₃ is chosen from hydrogen, halogen and methoxy;
R₄ is hydrogen or halogen;
R₆ is hydrogen or methyl;
R₇ is hydrogen, hydroxy or methoxy.

90. The method of claim 88, wherein the composition comprises from about 0.1% to about 99% by weight of the compound of Formula I or a pharmaceutically acceptable salt thereof.

91. The method of claim 88, wherein the composition comprises from about 10% to about 60% by weight of the compound of Formula I or a pharmaceutically acceptable salt thereof.
